SEQUENCE LISTING (14 sequences)

SEQ ID NO: 1 - protein, 836 aa, Homo Sapiens
MTTLAGAVPRMMRPGPGQNYPRSGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENPGMFSWEIRDKLLKDAVCDRNTVPSVSSISRILRSKFGKGEEEEADLERKEAEESEKKAKHSIDGILSERASAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPTAMPTLPTYQLSETSYQPTSIPQAVSDPSSTVHRPQPLPPSTVHQSTIPSNPDSSSAYCLPSTRHGFSSYTDSFVPPSGPSNPMNPTIGNGLSPQNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 2 - protein, 828 aa, Homo sapiens
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSGLVSSISRVLRIKFGKKEEEDEADKKEDDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 3 - DNA, 20 bp, Artificial Sequence
  PCR primer for the hg38 region of PAX3-FOXO1
cagaaccatcccattctccg

SEQ ID NO: 4 - DNA, 20 bp, Artificial Sequence
  PCR primer for hg38 region of PAX3-FOXO1
gcctgaccttgaacgtgaat

SEQ ID NO: 5 - DNA, 20 bp, Artificial Sequence
  PCR primer for hg38 region of PAX3-FOXO1
ggatcgtgtaacctgagggc

SEQ ID NO: 6 - DNA, 20 bp, Artificial Sequence
  PCR primer for hg38 region of PAX3-FOXO1
cagtgagtgtctgcctgcaa

SEQ ID NO: 7 - DNA, 20 bp, Artificial Sequence
  PCR primer for hg38 region of PAX3-FOXO1
tgcatgctggtgacagatca

SEQ ID NO: 8 - DNA, 20 bp, Artificial Sequence
  PCR primer for hg38 region of PAX3-FOXO1
acagcgtcctctttcagctt

SEQ ID NO: 9 - DNA, 22 bp, Artificial Sequence
  PCR primer for hg38 region of PAX3-FOXO1
ttcccatgaaaggagaagcaga

SEQ ID NO: 10 - DNA, 21 bp, Artificial Sequence
  PCR primer for hg38 region of PAX3-FOXO1
ggaaatgcctccacagaaacg

SEQ ID NO: 11 - DNA, 21 bp, Artificial Sequence
  PCR primer for hg38 region of PAX3-FOXO1
aaatttgaccttcgtcggcac

SEQ ID NO: 12 - DNA, 20 bp, Artificial Sequence
  PCR primer for hg38 region of PAX3-FOXO1
cagctgttggcgatttcacg

SEQ ID NO: 13 - DNA, 20 bp, Artificial Sequence
  PCR primer for hg38 region of PAX3-FOXO1
gctcttggttctctgtccct

SEQ ID NO: 14 - DNA, 20 bp, Artificial Sequence
  PCR primer for hg38 region of PAX3-FOXO1
agacagactgtgatgtgggg

(19) United States
(12) Patent Application Publication
(10) Pub. No.: US 2023/0365637 A1
(43) Pub. Date: Nov. 16, 2023

(54) IDENTIFICATION OF PAX3-FOXO1 BINDING GENOMIC REGIONS

(71) Applicant: The Research Institute at Nationwide Children's Hospital, Columbus, OH (US)

(72) Inventors: Benjamin Stanton, Bexley, OH (US); Benjamin Sunkel, Columbus, OH (US); Meng Wang, Columbus, OH (US)

(21) Appl. No.: 18/028,865

(22) PCT Filed: Sep. 28, 2021
(86) PCT No.: PCT/US21/52330

§ 371 Date: Mar. 28, 2023

Related U.S. Application Data
(60) Provisional application No. 63/084,098, filed on Sep. 28, 2020.

(51) Int. Cl. C07K 14/47; C12Q 1/6804; A61P 35/00; C12N 15/113

(52) U.S. Cl. CPC C07K 14/4702; C12Q 1/6804; A61P 35/00; C12N 15/113

(57) ABSTRACT

A method of identifying a plurality of regions in a genome that bind to PAX3-FOXO1 is described. The method includes the steps of obtaining chromatin from a cell; sonicating the chromatin; isolating the chromatin by immunoprecipitation using an antibody that binds to FOXO1, purifying the DNA from the immunoprecipitated chromatin; amplifying and sequencing the DNA; and analyzing the sequenced DNA to identify the regions in the genome of the cell that bind to PAX3-FOXO1. A method of treating a subject having rhabdomyosarcoma by modulating the expression of a genomic region of a cancer cell identified by the method is also described.

### CROSS-REFERENCE TO RELATED APPLICATIONS

This application claims priority from U.S. Provisional Application Ser. No. 63/084,098, filed Sep. 28, 2020, which is incorporated herein by reference.

### SEQUENCE LISTING

The instant application contains a Sequence Listing which has been submitted electronically in ASCII format and is hereby incorporated by reference in its entirety. Said ASCII copy, created on Sep. 22, 2021, is named NCH-029706 WO ORD SEQUENCE LISTING_ST25 and is 17,105 bytes in size.

### BACKGROUND

The pioneer factor subset of the broad transcription factor (TF) class of proteins possesses conserved motifs common to TFs, including a structured DNA-binding domain (DBD) responsible for motif recognition and a flexible transactivation domain mediating regulated recruitment (Ptashne and Gann, 1997). Distinct structural elements of pioneer factors (PFs) provide a unique capacity for high-affinity DNA binding despite steric deterrents at the chromatin interface, including nucleosomes. However, the combinatorial rules or “logic” of domain organization within PFs, requisite to achieve nucleosomal-motif recognition, remain obscure (Fernandez Garcia et al., 2019). Moreover, it is presently unknown if two pioneer factors fused together will possess pioneer activity in the resulting chimera.

Among characterized PFs, the forkhead family has been studied extensively at the structural and regulatory levels in mammalian development and also in multiple cancers (Herman et al., 2021). A winged-helix DNA-binding domain is conserved across individual members of this protein family In the case of FOXA1, the winged-helix domain mimics the structural features of the linker histone H1, disrupting H1-compacted chromatin together with the FOXA1 C-terminal domain (Cirillo et al., 1998, 2002; Zhou et al., 2020). Consistent with its high degree of sequence and structural similarity, the FOXO1 protein also recognizes its cognate DNA sequence motif within H1-compacted nucleosome arrays, initiating local DNase hypersensitivity through disruption of histone:DNA contacts without input from chromatin remodelers (Hatta and Cirillo, 2007). Of importance, chromatin decompaction following nucleosomal motif recognition by PFs is not necessarily associated with nucleosome eviction (Cirillo et al., 2002; Hatta and Cirillo, 2007). In a recently reported example, FOXA2 binding was shown principally to mediate the induction of nucleosome spacing within tissue-specific cis-regulatory regions (Iwafuchi-Doi et al., 2016). Findings such as these reinforce that PFs are capable of decompacting and stably binding to nucleosome-occupied regions of the genome, whereas recruitment of additional factors may be necessary for the formation of active and accessible regulatory elements generally associated with gene activation.

While evidence for direct nucleosomal motif recognition by putative PFs continues to emerge (Fernandez Garcia et al., 2019; Zhu et al., 2018), additional compact chromatin binding behaviors of PFs such as heterochromatin recognition and mitotic chromatin bookmarking are emerging from cell imaging studies. In addition to forkhead factors FOXI1 and FOXA1 (Yan et al., 2006; Zaret et al., 2008), other pioneer factors including SOX2, OCT4, and PAX3 are retained on compact mitotic chromatin (Deluz et al., 2016; Teves et al., 2016; Wu et al., 2015). In the case of SOX2, this may serve to mark specific genes for post-mitotic reactivation, whereas mitotic chromatin binding by PAX3 may instead be related to its reported function in the stable repression of microsatellite transcription via establishment and maintenance of H3K9me3-marked heterochromatin domains across cell divisions (Bulut-Karslioglu et al., 2012). These fundamental molecular functions of PFs likely underlie their central role in de novo activation of lineage-defining gene expression programs during tissue differentiation and contribute to heritable transmission of these gene programs during development.

In alveolar rhabdomyosarcoma, two pioneer factors, PAX3 and FOXO1, are fused in-frame in the recurrent translocation between chromosome arms 2p and 13q (Galili et al., 1993). The resulting PAX3-FOXO1 fusion is an oncogenic driver that has been described as binding active regulatory elements alongside myogenic TFs (Gryder et al., 2019), whereas its nucleosome targeting function in inactive or repressed chromatin domains remains unstudied. Neither retention of canonical pioneer activity nor the emergence of functions distinct from the wild-type PAX3 or FOXO1 monomers has been rigorously defined for PAX3-FOXO1 in fusion-positive rhabdomyosarcoma (FP-RMS). Given the relatively low mutational frequencies in FP-RMS, which can be approximated at 0.1 protein-coding mutations per Mb (Shern et al., 2014), the inventors hypothesized that the pioneer function of PAX3-FOXO1, defined by targeting to nucleosomal motifs within inaccessible chromatin, might underlie its transforming potential in this tumor. However, the mechanisms through which PAX3-FOXO1 engages distinct classes of chromatin have remained poorly understood.

The cascade of initiation events for a tumor are unlikely to involve mere stabilization of pre-existing transcriptional networks or DNA accessibility, but rather, a restructuring of regulatory elements into a new state that differs from a cell of origin. Presently, the cell of origin for FPRMS remains unknown. Expression of PAX3-FOXO1, along with other highly expressed TFs in FP-RMS, is reminiscent of both neuronal tissue and developing muscle (Galili et al., 1993), contributing to ambiguity in defining a tissue of origin. Often, transcriptional reprogramming represents the functional output of tissue-specific pioneer factors. It is noteworthy that tumors prevalent in children are frequently defined by a profound failure of cellular differentiation (Nacev et al., 2020). In these and other relatively low-mutation-burden tumors, there has been increasing evidence suggesting that disruption of transcription factors drives reprogramming into altered epigenetic states. The role of PFs like SOX2, PAX3, and FOXO1 in developmental reprogramming may be analogous to pioneer activity in establishing cell-fate decisions in pediatric cancers, including synovial sarcoma and MPNST (Kadoch and Crabtree, 2013; Miller et al., 2009), where mis-regulation of SOX-family pioneer factors occurs, as well as in FP-RMS, where PAX3 and FOXO1 are frequently fused as a chimeric oncoprotein.

Rhabdomyosarcoma (RMS) is a devastating pediatric cancer with the most aggressive form of the disease being genetically defined by fusions between PSX3/7 and FOXO1. This rare pediatric tumor has a poor prognosis, with survival rates at 30-50%, that have not improved in several decades. In fusion-positive RMS (FP-RMS), the early targeting function of the primary fusion protein PAX3-FOXO1 has remained unclear. Accordingly, there has been a critical need to precisely define the requirements for PAX3-FOXO1 function at the chromatin level. PAX3-FOXO1 uses super enhancers to set up autoregulatory loops in collaboration with the master transcription factors MYOG, MYOD, and MYCN. Gryder et al., 2017. However, the immediate targeting mechanisms of PAX3-FOXO1 in the context of chromatin accessibility have yet to be assessed in a temporally-controlled system.

Therapy for the aggressive alveolar RMS subtype relies upon surgery, radiation, and broadly toxic drugs. Arndt et al., 2009. Understanding the immediate localization of the driving translocation in FP-RMS is critical for identifying new targetable genes under the control of PAX3-FOXO1.

### SUMMARY OF THE INVENTION

The inventors and their colleagues have recently discovered that PAX3-FOXO1 accumulates in the soluble euchromatic nuclear fractions and in the insoluble heterochromatic pellet with ammonium sulfate nuclear extractions, while wildtype FOXO1 accumulates in the cytoplasm. This has several interesting implications. First, either PAX3-FOXO1 forms insoluble condensates due to some intrinsic disorder, or binds outside of euchromatic regions, or that FOXO1 is subject to active nuclear export in the presence of the fusion protein.

The inventors speculated that nuclear FOXO1 epitopes in FP-RMS cells would be present only in the context of the PAX3-FOXO1 fusion. They therefore carried out a ChIP-seq analysis using a FOXO1 antibody that recognizes the C-terminal region that is preserved after translocation. The inventors found that “under-sonicating” the chromatin preserved many binding sites that may have been missed in previous studies of PAX3-FOXO1 localization. Their study produced 9,063 binding sites enriched with the previously characterized PAX3-FOXO1 binding motif which overlapped 69% of PAX3-FOXO1 sites identified in previous localization studies. Cao et al., Cancer Res. 70, 6497-6508 (2010). The biological replicates for each sample were then sequenced. The 7,282 unique PAX3-FOXO1 binding sites, mapping to thousands of inactive genomic loci outside of enhancers, represent a new paradigm in the understanding of targeting by PAX3-FOXO1.

The inventors have identified a new method to (1) localize PAX3-FOXO1 across the genome, (2) define its biochemical fractionation, and (3) operationalize an inducible system for rapid regulation. This work enables the definition of immediate-early target loci for PAX3-FOXO1, the most common oncogenic driver for FP-RMS, which expands the scope of actionable targets for this type of cancer.

### BRIEF DESCRIPTION OF THE FIGURES

The present invention may be more readily understood by reference to the following figures, wherein:

FIG. 1 provides a schematic representation of the pioneer factor fusion oncoprotein in Rhabdomyosarcoma, the PAX-FOXO1 steady state system, and the PAX3-FOXO1 induction system.

FIGS. 2A-2I provide graphs and figures showing cellular and genomic localization of the PAX3-FOXO1 fusion transcription factor to inactive chromatin. (A) Evolutionary conservation of PAX3 (top) and FOXO1 (bottom) amino acid sequences. Vertical dashed lines indicate the breakpoint position resulting in the formation of the PAX3-FOXO1 fusion. FOXO1 inset details N-terminal truncation of the winged helix domain. (B) FN-RMS (RD, SMS-CTR) and FP-RMS (RH4, RH30) cells were fractionated to interrogate protein localization within the cytoplasm, soluble nucleus, soluble chromatin (sonication sensitive), and chromatin pellet (sonication insensitive) compartments of the cell. (C) A C-terminal FOXO1 antibody was utilized to perform per-cell ChIP-seq (pc-ChIP-seq) of the PAX3-FOXO1 fusion oncogene in a panel of RMS cells. Spike-in normalized signal intensity from each cell line was displayed across the union of PAX3-FOXO1 sites identified in RH4 and RH30 cells. (D) HOMER motif analysis was performed on PAX3-FOXO1 binding sites from anti-FOXO1 pc-ChIP-seq (RH4 and RH30), and pFM2 ChIP-seq (RH4, Cao et al., Cancer Res 2010). RNA-seq expression for the transcription factor corresponding to each motif is displayed as the median expression for PAX3-FOXO1+ alveolar RMS (ARMS) cells in the Cancer Cell Line Encyclopedia (CCLE). The PAX3-FOXO1 motif consists of a Paired Domain (PD) and a Homeobox Domain (HD) motif arranged in a divergent, head-to-head orientation. Representative PDB structures demonstrate the recognition of PD and HD motifs by short alpha-helices, where E-box motifs are recognized by the extended alpha-helices of bHLH family transcription factors. (E) ATAC-seq was performed in RH4 cells, and Tn5 insertion positions were calculated by the NucleoATAC pipeline. Insertion rates are displayed with respect to high confidence PAX3-FOXO1 (left) and FOXO1 (right) motif positions (vertical dashed lines) within PAX3-FOXO1 binding sites identified using FIMO. The median insertion rate outside and within the motif positions is displayed as a black and red horizontal line, respectively. (F and G) (F) Heatmap of PAX3-FOXO1, histone marks, and ATAC-seq in P3F-binding sites categorized as promoters, enhancers, clustered enhancers, and non-enhancers in RH4 cells. (G) Analysis of H3K27ac and P3F in enhancer clusters. (Top, Middle) H3K27ac (Top) and P3F (Middle) signal over clustered enhancer regions with or without an overlapping P3F-binding site. (Bottom) P3F and H3K27ac signal centered over P3F-binding sites occurring within enhancer clusters. (H) H3K9me3 ChIP-seq was performed in RH4 cells with a modified library preparation. H3K9me3 profiles were characterized over P3F-binding sites in promoters, enhancers, clustered enhancers, and non-enhancers. Ordering P3F sites according to the degree of 50-30 H3K9me3 signal imbalance, H3K9me3 signals were found to exhibit asymmetry around P3F-binding sites. (I) H3K27ac-marked enhancer regions were divided into three categories displaying (1) no flanking H3K9me3, (2) intermediate flanking H3K9me3, or (3) high flanking H3K9me3. H3K27ac, H3K9me3, and P3F profile plots were generated for each enhancer category. Up- and downstream H3K9me3 imbalance were significantly higher in “high flanking H3K9me3” sites than in “intermediate flanking H3K9me3” sites (Upstream p value=1×10⁻⁷, Downstream p value=0) and “no flanking H3K9me3” sites (Upstream p=0, Downstream p value=0) (ANOVA, Tukey's HSD correction).

FIGS. 3A-3G provide graphs and images showing the initial genomic targeting of PAX3-FOXO1 exhibits nucleosomal motif recognition. (A) PAX3-FOXO1 peaks called in Dbt/iP3F cells following treatment with 500 ng/mL doxycycline for 0, 8, or 24 hours. Motif analysis results are displayed for the 8 and 24 hour timepoints. (B) Spike-in normalized signal plot for PAX3-FOXO1 over the union of peaks identified at 0, 8, and 24 hours. (C) ATAC-seq data from Dbt/iP3F cells treated with doxycycline for 0, 8, and 24 hours was utilized for k-means clustering (k=3) of induced PAX3-FOXO1 binding sites. Local enrichment of ATAC-seq signals (calculated as the fold change of the ATAC signal within binding sites vs. 10X-sized background regions immediately flanking each binding site) is displayed for PAX3-FOXO1 binding site Groups 1, 2, and 3. (D) Normalized ChIP-seq signal intensity and accessibility plotted across equilibrium PAX3-FOXO1 binding site Clusters 1, 2, and 3. (E) NucleoATAC was performed using time course ATAC-seq data in Dbt/iP3F cells across equilibrium PAX3-FOXO1 binding site Clusters 1, 2, and 3. Inferred nucleosome occupancy is displayed with respect to PAX3-FOXO1 peaks. (F and G) (F) NucleoATAC of Enhancer and non-enhancer P3F-binding sites in RH4 cells. (G) Plot2DO analysis of ATACseq fragment lengths present within induced PAX3-FOXO1-binding sites in Dbt/iP3F cells following 0, 8, and 24 h doxycycline treatment.

### DETAILED DESCRIPTION OF THE INVENTION

In one aspect, the present invention provides a method of identifying a plurality of regions in a genome that bind to PAX3-FOXO1. The method includes the steps of obtaining chromatin from a cell; sonicating the chromatin; isolating the chromatin by immunoprecipitation using an antibody that binds to FOXO1, purifying the DNA from the immunoprecipitated chromatin; amplifying and sequencing the DNA; and analyzing the sequenced DNA to identify the regions in the genome of the cell that bind to PAX3-FOXO1. Another aspect of the invention provides a method of treating a subject having rhabdomyosarcoma by modulating the expression of a genomic region of a cancer cell identified by the method.

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise.

Throughout this application, the term “about” is used to indicate that a value includes the standard deviation of error for the device or method being employed to determine the value, except that the value will never deviate by more than 5% from the value cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

“Treating”, as used herein, means ameliorating the effects of, or delaying, halting or reversing the progress of a disease or disorder. Treatment includes prophylactic treatment of subjects diagnosed with cancer who have not yet exhibited symptoms of the disease, and non-prophylactic treatment of subjects who have exhibited symptoms. The word encompasses reducing the severity of a symptom of a disease or disorder and/or the frequency of a symptom of a disease or disorder. A subject is successfully “treated” for a disease or disorder if the subject shows observable and/or measurable reduction in or absence of one or more signs and symptoms of a particular disease or condition.

A “subject”, as used therein, can be a human or non-human animal Non-human animals include, for example, livestock and pets, such as ovine, bovine, porcine, canine, feline and murine mammals, as well as reptiles, birds and fish. Preferably, the subject is human Subjects can also be selected from different age groups. For example, the subject can be a child, adult, or elderly subject.

The term “gene,” as used herein, means one or more sequence(s) of nucleotides in a genome that together encode one or more expressed molecule, e.g., an RNA, or polypeptide. The gene can include coding sequences that are transcribed into RNA which may then be translated into a polypeptide sequence, and can include associated structural or regulatory sequences that aid in replication or expression of the gene.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. The term “nucleotide sequence,” as used herein, refers to an oligonucleotide, nucleotide, or polynucleotide of single-stranded or double stranded DNA or RNA, or fragments thereof.

DNA (deoxyribonucleic acid), as is understood by those skilled in the art, is a molecule consisting of two long polymers of simple units called nucleotides with a backbone made of alternating sugars (deoxyribose) and phosphate groups that forms a double-stranded helix. The nucleotides include guanine, adenine, thymine, and cytosine, which are referenced using the letters G, A, T, and C.

The term “antibody” as used herein refers to immunoglobulin molecules or other molecules which comprise at least one antigen-binding domain. The term “antibody” as used herein is intended to include whole antibodies, monoclonal antibodies, polyclonal antibodies, chimeric antibodies, humanized antibodies, primatized antibodies, multi-specific antibodies, single chain antibodies, epitope-binding fragments, e.g., Fab, Fab′ and F(ab′)2, Fd, Fvs, single-chain Fvs (scFv), disulfide-linked Fvs (sdFv), fragments comprising either a VL or VH domain, and totally synthetic and recombinant antibodies. The antibodies can be of any type (e.g., IgG, IgE, IgM, IgD, IgA, and IgY), class (e.g., IgG1, IgG2, IgG3, IgG4, IgA1 and IgA2) or subclass of immunoglobulin molecule.

### Identifying PAX3-FOXO1 Binding Regions

In one aspect, the present invention provides a method of identifying a plurality of regions in a genome that bind to PAX3-FOXO1. Typically the method is performed as part of a chromatin immunoprecipitation (ChIP) assay. The term “chromatin immunoprecipitation assay” is well known to one skilled in the pertinent art, and preferably comprises at least the following steps: (i) preparation of a liquid sample comprising chromatin to be analyzed from cells; (ii) immunoprecipitation of the chromatin in the liquid sample onto the matrix using an antibody; (iii) DNA recovery from the precipitated chromatin; and (iv) DNA analysis.

One step of the method includes obtaining chromatin from a cell. Chromatin consists of a complex of DNA and protein (primarily histone) and makes up the chromosomes found in eukaryotic cells. Chromatin occurs in two states, euchromatin and heterochromatin, with different staining properties, and during cell division it coils and folds to form the metaphase chromosomes. Chromatin is used herein to refer to any such complex of nucleic acid (typically DNA) and associated proteins, including chromatin fragments produced by fragmentation of chromosomes or other chromatin preparations.

The cells evaluated by the method can be cancer cells, such as rhabdomyosarcoma cells. Typically, the method may be performed on a sample comprising chromatin from 10³ to 10⁹ cells, e.g. preferably less than 10⁷ cells, less than 10⁶ cells or less than 10⁵ cells, preferably about 10⁴ to 10⁶ cells. One cell typically contains about 6 pg (6×10⁻¹² g) DNA per cell and equal amounts of DNA and protein in chromatin. Thus, the method may be performed, for example, on a sample comprising about 0.6 μg DNA, or 1.2 μg of chromatin (this equates to mass of DNA or chromatin in about 100,000 cells). In some embodiments, the chromatin is obtained from at least 1,000 cells.

The method can also include the step of the step of obtaining the cells from a subject. Alternately, in some embodiments, the cells may have already been obtained. Cells can be obtained from subjects for diagnosis prognosis, monitoring, or a combination thereof, or for research, or can be obtained from un-diseased individuals, as controls or for basic research.

In another embodiment, the method may comprise a step of cross-linking the chromatin before obtaining it from the cell. This may be achieved for any suitable means, for example, by addition of a suitable cross-linking agent, such as formaldehyde, preferably prior to fragmentation of the chromatin. Formaldehyde crosslinking can be used for the detection and quantification of protein-DNA interactions or the interactions between chromatin proteins. See Hoffman et al., 2015. Additional suitable protein-DNA cross-linking agents are known to those skilled in the art. Fragmentation may be carried out by sonication. However, formaldehyde may be added after fragmentation, and then followed by nuclease digestion. Alternatively, UV irradiation may be employed as an alternative cross-linking technique.

In one embodiment, cells or tissue fragments are first fixed with formaldehyde to crosslink protein-DNA complex. Cells can be incubated with formaldehyde at room temperature or at 37° C. with gentle rocking for 5-20 min, preferably for 10 min. Tissue fragments may need a longer incubation time with formaldehyde, for example 10-30 min, e.g. 15 min. The concentration of formaldehyde can be from 0.5 to 10%, e.g. 1% (v/v).

Once the crosslinking reaction is completed, an inhibitor of crosslink agents such as glycine at a molar concentration equal to crosslink agent can be used to stop the crosslinking reaction. An appropriate time for stopping the crosslinking reaction may range from 2-10 min, preferably about 5 min at room temperature. Cells can then be collected and lysed with a lyses buffer containing a sodium salt, EDTA, and detergents such as SDS. Tissue fragments can be homogenized before lysing.

Chromatin is then extracted from the preparation comprising cells to prepare a liquid sample comprising chromatin fragments. Cells or the homogenized tissue mixture can be mechanically or enzymatically sheared to yield an appropriate length of the DNA fragment. Usually, 200-1000 base pairs of sheared chromatin or DNA is required for the ChIP assay. Mechanical shearing of DNA can be performed by nebulization or sonication, preferably sonication. Enzymatic shearing of DNA can be performed by using DNAse I in the presence of Mn salt, or by using micrococcal nuclease in the presence of Mg salt to generate random DNA fragments. The conditions of crosslinked DNA shearing can be optimized based on cells, and sonicator equipment or digestion enzyme concentrations.

In some embodiments, the chromatin is obtained from the cells using sonication. The inventors have discovered that under-sonicating the preparation including the cells can increase the number of genomic regions that are identified by the method. Under-sonicating refers to sonicating the chromatin preparation for less time than one skilled in the art would normally sonicate the chromatin preparation. In some embodiments, under-sonicating refers to sonicating the chromatin preparation for less than half of the time that would normally used by one skilled in the art. In further embodiments, the chromatin preparation is sonicated for less than 30 minutes, less than 25 minutes, less then 20 minutes, less than 15 minutes, less than 10 minutes, or less than 5 minutes.

The inventors have also discovered that incubation of the sonicated chromatin in a salt buffer having a higher than normal concentration can improve the performance of the method. A variety of different buffers suitable for use in ChiP can be used. In some embodiments, the buffer includes a salt having a concentration that is at least 10%, 20%, 30%, 40%, 50%, 60%, 70%, 80%, 90%, 100%, 125%, 150%, or 200% higher than the normal salt concentration used for the ChiP buffer. In some embodiments, a concentration of a salt (e.g., sodium chloride) of at least 150 mM, at least 175 mM, or at least 200 mM can be used.

In some embodiments, once DNA shearing is completed, cell debris can be removed by centrifugation, and supernatant containing DNA-protein complex is collected. The result is a liquid sample comprising chromatin fragments in which the protein is immobilized on the DNA (e.g. wherein the DNA and protein are cross-linked). In an alternative embodiment, the centrifugation step may be omitted, i.e. the following steps are performed directly after DNA shearing.

Once the proteins have been immobilized on the chromatin, the PAX3-FOXO1-DNA complex may then be immunoprecipitated. Hence, once the sample comprising chromatin has been prepared, the method includes the step of immunoprecipitating the chromatin. Preferably immunoprecipitation is carried out by addition of a suitable antibody that binds, or specifically binds, to FOXO1.

Antibodies are designed for specific binding, as a result of the affinity of complementary determining region of the antibody for the epitope of the biological analyte (in this case, FOXO1). An antibody “specifically binds” when the antibody preferentially binds a target structure, or subunit thereof, but binds to a substantially lesser degree or does not bind to a biological molecule that is not a target structure. In some embodiments, the antibody specifically binds to the target analyte with a specific affinity of between 10⁻⁸ M and 10⁻¹¹ M. In some embodiments, an antibody or antibody fragment binds to the target analyte with a specific affinity of greater than 10⁻⁷ M, 10⁻⁸ M, 10⁻⁹ M, 10⁻¹⁰ M, or 10⁻¹¹ M, between 10⁻⁸ M-10⁻¹¹ M, 10⁻⁹ M-10⁻¹⁰ M, and 10⁻¹⁰ M-10⁻¹¹ M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

Protocols for generating antibodies, including preparing immunogens, immunization of animals, and collection of antiserum may be found in Antibodies: A Laboratory Manual, E. Harlow and D. Lane, ed., Cold Spring Harbor Laboratory (Cold Spring Harbor, N.Y., 1988) pp. 55-120 and A. M. Campbell, Monoclonal Antibody Technology: Laboratory Techniques in Biochemistry and Molecular Biology, Elsevier Science Publishers, Amsterdam, The Netherlands (1984). Monoclonal antibodies may be produced in animals such as mice and rats by immunization. B cells can be isolated from the immunized animal, for example from the spleen. The isolated B cells can be fused, for example with a myeloma cell line, to produce hybridomas that can be maintained indefinitely in in vitro cultures.

For location studies of fusion transcription factors such as those described herein, the antibody used should be raised against the amino acid residues of the translocation partners that are preserved within the final fusion protein. Preferably, the antibody used is either against a portion of the fusion protein having a wild type having sufficiently low abundance compared to the fusion protein as a whole, or the antibody performs well for localization of the fusion protein, but not for localization of the wild type portion of the fusion protein.

The antibody used should bind, or specifically bind, to the PAX3-FOXO1 fusion protein. In some embodiments, the antibody specifically binds to the FOXO1 region of the fusion protein, while in other embodiments the antibody specifically binds to the PAX3 region of the fusion protein. In some embodiments, the antibody is the Cell Signaling Catalog #2880 antibody, which binds to wild-type FOXO1. This is a rabbit monoclonal antibody raised against a GST-fusion peptide corresponding to the carboxy-terminal residues of human FOXO1. It is a knockout-validated antibody described by Deng et al. Deng et al., 2012. It is commercially available from Cell Signaling Technology®, Danvers, MA.

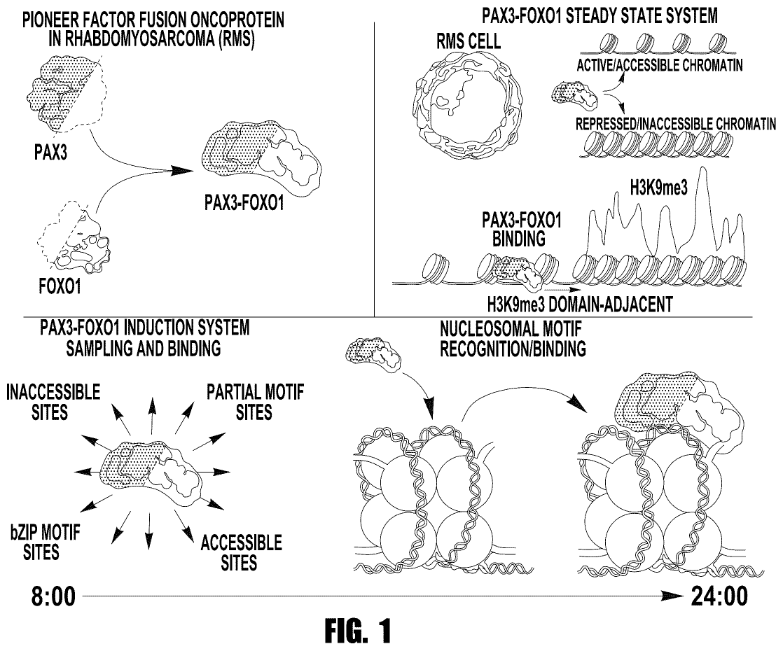

PAX3-FOXO1 is an oncogenic, chimeric transcription factor. An overview of roles played by PAX-FOXO1 is provided by FIG. 1. Transcription factor, PAX3 plays an essential role in myogenesis. A recurrent chromosomal translocation results in the formation of a gene fusion, PAX3-FOXO1. The fusion consists of the first seven exons of PAX3 and the last two exons of FOXO1. The fusion breakpoint typically occurs between exon 7 of the PAX3 coding sequence and exon 2 of FOXO1, although distinct breakpoints are observed in individual rhabdomyoscarcoma patients and cell lines. However, because the antibody used is typically against an invariable amino acid sequence in RMS cells and patient tumors (i.e., the FOXO1 C-terminus), it should perform well regardless of the specific breakpoint position. The inventors have demonstrated this by performing PAX3-FOXO1 ChIP-seq in both RH4 and RH30 cells, which have different breakpoints.

The amino acid sequence of PAX3-FPXP1 in RH4 cells is provided below:

(SEQ ID NO: 1)
MTTLAGAVPRMMRPGPGQNYPRSGFPLEVSTPLGQGRVNQLGGVFINGR
PLPNHIRHKIVEMAHHGIRPCVISRQLRVSHGCVSKILCRYQETGSIRP
GAIGGSKPKQVTTPDVEKKIEEYKRENPGMFSWEIRDKLLKDAVCDRNT
VPSVSSISRILRSKFGKGEEEEADLERKEAEESEKKAKHSIDGILSERA
SAPQSDEGSDIDSEPDLPLKRKQRRSRTTFTAEQLEELERAFERTHYPD
IYTREELAQRAKLTEARVQVWFSNRRARWRKQAGANQLMAFNHLIPGGF
PPTAMPTLPTYQLSETSYQPTSIPQAVSDPSSTVHRPQPLPPSTVHQST
IPSNPDSSSAYCLPSTRHGFSSYTDSFVPPSGPSNPMNPTIGNGLSPQN
SIRHNLSLHSKFIRVQNEGTGKSSWWMLNPEGGKSGKSPRRRAASMDNN
SKFAKSRSRAAKKKASLQSGQEGAGDSPGSQFSKWPASPGSHSNDDFDN
WSTFRPRTSSNASTISGRLSPIMTEQDDLGEGDVHSMVYPPSAAKMAST
LPSLSEISNPENMENLLDNLNLLSSPTSLTVSTQSSPGTMMQQTPCYSF
APPNTSLNSPSPNYQKYTYGQSSMSPLPQMPIQTLQDNKSSYGGMSQYN
CAPGLLKELLTSDSPPHNDIMTPVDPGVAQPNSRVLGQNVMMGPNSVMS
TYGSQASHNKMMNPSSHTHPGHAQQTSAVNGRPLPHTVSTMPHTSGMNR
LTQVKTPVQVPLPHPMQMSALGGYSSVSSCNGYGRMGLLHQEKLPSDLD
GMFIERLDCDMESIIRNDLMDGDTLDFNFDNVLPNQSFPHSVKTTTHSW
VSG.

In addition to PAX3-FOXO1 fusion proteins, PAX7-FOXO1 fusions are also formed in RMS by the translocation of exon 7 in PAX7 with exon 2 in FOXO1. An additional representative amino acid sequence of a PAX7-FOXO1 fusion is:

(SEQ ID NO: 2)
MAALPGTVPRMMRPAPGQNYPRTGFPLEVSTPLGQGRVNQLGGVFINGR
PLPNHIRHKIVEMAHHGIRPCVISRQLRVSHGCVSKILCRYQETGSIRP
GAIGGSKPRQVATPDVEKKIEEYKRENPGMFSWEIRDRLLKDGHCDRST
VPSGLVSSISRVLRIKFGKKEEEDEADKKEDDGEKKAKHSIDGILGDKG
NRLDEGSDVESEPDLPLKRKQRRSRTTFTAEQLEELEKAFERTHYPDIY
TREELAQRTKLTEARVQVWFSNRRARWRKQAGANQLAAFNHLLPGGFPP
TGMPTLPPYQLPDSTYPTTTISQDGGSTVHRPQPLPPSTMHQGGLAAAA
AAADTSSAYGARHSFSSYSDSFMNPAAPSNHMNPVSNGLSNSIRHNLSL
HSKFIRVQNEGTGKSSWWMLNPEGGKSGKSPRRRAASMDNNSKFAKSRS
RAAKKKASLQSGQEGAGDSPGSQFSKWPASPGSHSNDDFDNWSTFRPRT
SSNASTISGRLSPIMTEQDDLGEGDVHSMVYPPSAAKMASTLPSLSEIS
NPENMENLLDNLNLLSSPTSLTVSTQSSPGTMMQQTPCYSFAPPNTSLN
SPSPNYQKYTYGQSSMSPLPQMPIQTLQDNKSSYGGMSQYNCAPGLLKE
LLTSDSPPHNDIMTPVDPGVAQPNSRVLGQNVMMGPNSVMSTYGSQASH
NKMMNPSSHTHPGHAQQTSAVNGRPLPHTVSTMPHTSGMNRLTQVKTPV
QVPLPHPMQMSALGGYSSVSSCNGYGRMGLLHQEKLPSDLDGMFIERLD
CDMESIIRNDLMDGDTLDFNFDNVLPNQSFPHSVKTTTHSWVSG.

In some embodiments, a binding domain other than PAX3 is included in a fusion protein together with FOXO1. These fusion proteins are referred to herein as DNA Binding Domain-FOXO1 fusion proteins. FOXO1 fusions preserving the C-terminal amino acid sequence have been detected in stomach adenocarcinoma (WDFY2-FOXO1), lung adenocarcinoma (SMARCA4-FOXO1), and B-cell precursor acute lymphoblastic leukemia (MEIS1-FOXO1). The method of the invention is therefore generalizable to these and other FOXO1 fusions as well.

The DNA is then purified from the immunoprecipitated chromatin. Where the sample comprised crosslinked DNA-protein complexes, the crosslinking can be reversed after washing. The buffer for crosslink reversal can be optimized to maximize reversal of the crosslinks and minimize DNA degradation resulting from chemical, biochemical and thermodynamic action. For example, in one embodiment the buffer for reversal of crosslinking comprises EDTA, SDS, and proteinase K, which should efficiently degrade proteins complexed with DNA and prevent degradation of DNA by nucleases such as DNAse I. A further buffer may also be used comprising sodium and potassium salts with a high concentration, e.g. sodium chloride at 1M or potassium chloride at 0.5 M. Such buffers have been demonstrated to efficiently reduce DNA degradation from chemical and thermodynamic action (Marguet, E. Forturre, P, 1998) and increase the reversing rate of formaldehyde crosslinks. Typically reversal of crosslinking takes place at elevated temperature, e.g. 50-85° C. for 5 min-4 hours, preferably at 65-75° C. for 0.5-1.5 h.

Once reversal of the crosslinked DNA-protein complex has been completed, DNA may be captured and cleaned. This may be achieved by the standard technique of phenol-chloroform extraction, or by capturing DNA on a further solid phase (e.g. silica or nitrocellulose in the presence of high concentrations of non-chaotropic salts).

In some embodiments, rather than utilizing phenol-chloroform extraction for purification of ChIP and Input DNA samples, commercial reagents for silica gel-based spin-column purification can be used. In this method, DNA suspended in a buffer of alcohol and salts is passed through a silica gel membrane to which it binds via centrifugation. The DNA-bound membrane is washed to remove contaminants, and the DNA is finally eluted from the membrane in a low salt buffer or water. This method is efficient, convenient, and reduces exposure to harmful organic solvents.

Following the purification step, the isolated DNA fragments may then be amplified and analyzed to sequencing the DNA. This can be achieved using the polymerase chain reaction (PCR). For example, the analysis step may comprise use of suitable primers, which during PCR, will result in the amplification of a length of nucleic acid. The term “PCR” includes all variants of the technique commonly known to the person skilled in the art, including allele-specific PCR, dial-out PCR, digital PCR, hot-start PCR, inverse PCR, ligation-mediated PCR, methylation-specific PCR, mini-primer PCR, multiplex PCR, nano-PCR, nested PCR, quantitative PCR (qPCR), reverse-transcription PCR, solid phase PCR, and touchdown PCR. The skilled person will appreciate that the method may be applied to detect genes or any region of the genome for which specific PCR primers may be prepared. The PCR results may be viewed, for example, on an electrophoretic gel. qPCR would provide quantitative analysis of the DNA present and is the preferred form of PCR for this method. Other techniques that could be used are direct sequencing of the DNA fragments or microarray hybridization.

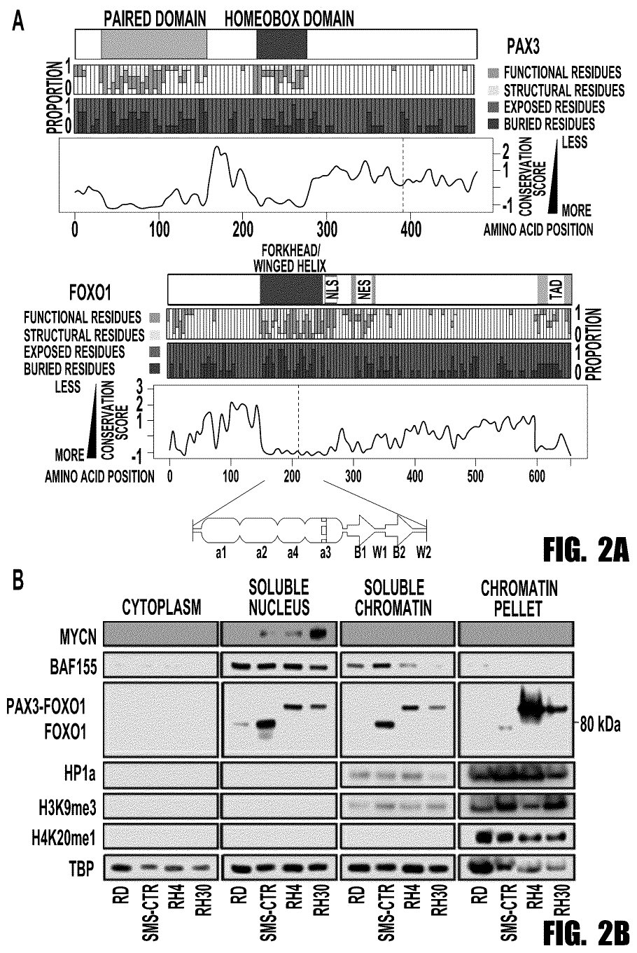

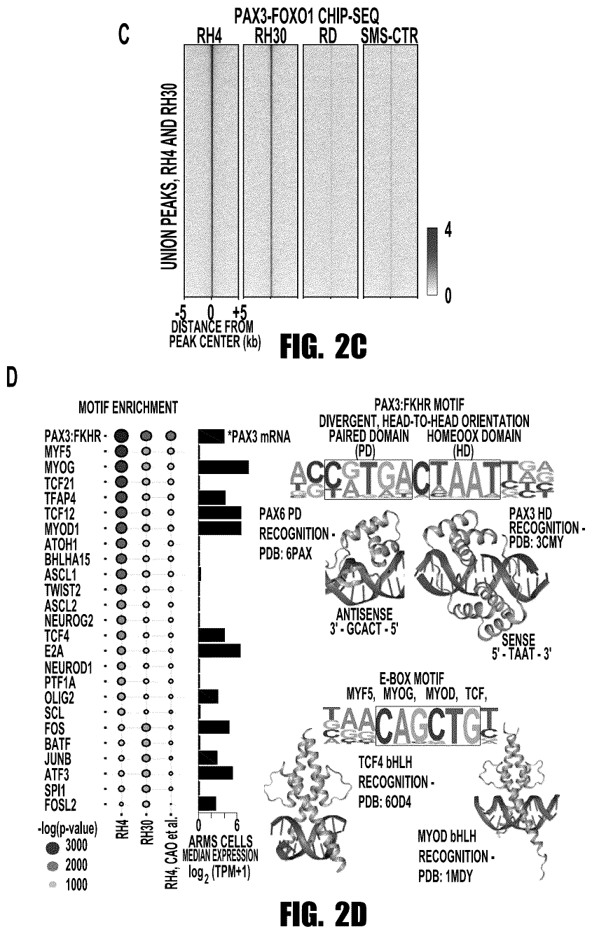

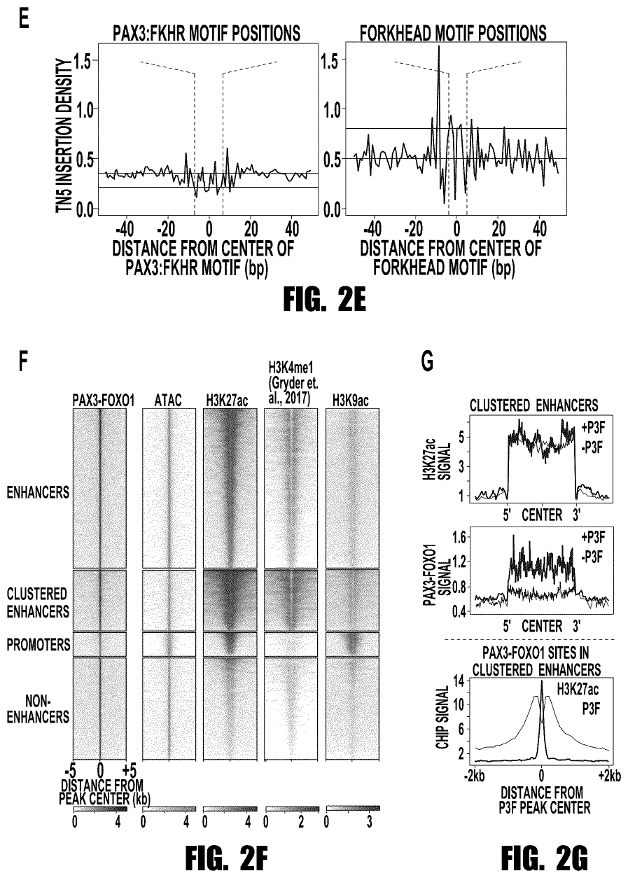

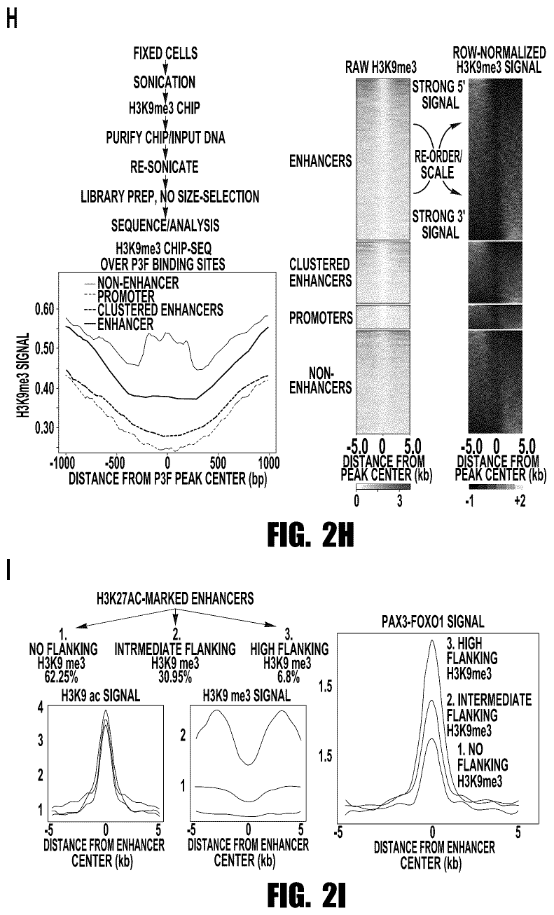

Typically, there are two uses of the polymerase chain reaction (PCR) in the method. The first is the use of qPCR for low-throughput validation or quality control of the ChIP sample after decrosslinking/purification. This precedes the preparation of a sequencing library, and it utilizes short oligonucleotide primers to amplify specific DNA sequences representing known PAX3-FOXO1-bound and -unbound sites in the genome. The results of this use of PCR are reflected in FIG. 2C & 2F.

The second use of PCR occurs in during the preparation of ChIP and Input DNA for high-throughput sequencing in a process called library generation. There are multiple methods for library generation of ChIP and Input DNA including tagmentation, template switching, and adaptor ligation. A generalized method of the adaptor ligation process generating libraries to be analyzed on various next-generation sequencing platforms includes the following steps:
1) DNA end repair, to eliminate single-stranded DNA overhangs and produce blunt ended DNA fragments.
2) A-tailing, to introduce a single 3′ deoxyadenosine (3′ dA) base to each end of each DNA fragment in the sample
3) Adaptor ligation, to append a double-stranded DNA fragment (adaptor) to each piece of DNA in the sample. The adaptor has a 3′ deoxythymidine base for complementation to the 3′ dA on DNA fragments in the sample.
4) Optional size selection, to isolate DNA fragments of a certain base pair length. Usually, a length range of 200-400 base pairs is used. This is often performed using gel electrophoresis, where the desired fragments are excised from the gel and purified via silica gel column-based extraction.
4) PCR amplification using primers that recognize DNA sequences within the adaptor present on DNA fragments in the sample. This step amplifies the adaptor-ligated DNA fragments and introduces additional sequences known as barcodes. With DNA fragments from individual samples/experiments containing a unique barcode, multiple samples may be combined for sequencing and data can be subsequently separated before final analysis.
5) Library purification removes excess PCR primers. This is often performed using gel electrophoresis or magnetic bead-based (e.g. AMPure XP) purification methods. Following these steps, libraries are ready for sequencing, in our case on an Illumina platform, to determine the sequence of each DNA fragment in the sample.

Once the DNA has been amplified and their sequences determined, the DNA is analyzed to identify the regions in the genome of the cell that bind to PAX3-FOXO1. The methods identify variably-sized sets of residues in genomes (i.e., genomic regions) that are bound by PAX-FOXO1. The genomic regions can include a range of base pairs. In some embodiments, the genomic region includes a number of base pairs ranging from 100 to 100,000, from 1000 to 100,000, from 5000 to 100,000, from 10,000 to 100,000, from 100 to 50,000, from 100 to 10,000, from 100 to 5,000, from 1000 to 50,000, or from 5,000 to 50,000. The genomic region includes genes and gene-sized polynucleotides.

The method has been demonstrated to identify more PAX3-FOXO1 binding sites than prior art methods. In some embodiments, the method can identify at least 1,000, at least 5000, at least 7500, at least 10,000, at least 12,500, or at least 15,000 genomic regions. In some embodiments, one or more of the genomic regions are a portion of a gene.

For sequence comparison and identification, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are typically used.

A number of specific methods are available for carrying out a sequence analysis. Short read alignment tools (e.g. bowtie, bowtie2, bwa) are utilized to align DNA sequence reads generated by the high-throughput DNA sequencing platform to a reference genome (e.g. hg38, mm10) using parameters discussed in the Example, herein.

Once reads are aligned, peak-calling algorithms (e.g. MACS, MACS2) are employed to identify regions of the genome where there is an abundance of reads accumulating using parameters discussed in the Example. These are designated as PAX3-FOXO1 binding sites when the number of reads aligning to a region in the ChIP sample exceeds the number of reads expected to align to that region (based on the Input sample) at a defined statistical threshold. Those skilled in the art recognize that there are many tools and options for performing the sequence analysis.

In some embodiments, the cell is a human cell, and the DNA is analyzed by alignment with a human genome sequence, such as UCSC hg38. In some embodiments, a “spike in” strategy is used, which permits downstream analysis of ChiP specificity using concurrent ChiP assays against cells from different species. For example, in some embodiments, the cells include human cells and rodent cells, wherein the rodent cells express wild-type FOXO1.

### Methods of Treating Rhabdomyoscarcoma

Another aspect of the invention provides a method of treating a subject having rhabdomyosarcoma. The method includes modulating the expression of a genomic region of a cancer cell identified by the method of analysis described herein.

Rhabdomyosarcoma is an aggressive and highly malignant form of cancer that develops from skeletal (striated) muscle cells that have failed to fully differentiate. It is generally considered to be a disease of childhood, as the vast majority of cases occur in those below the age of 18. Rhabdomyosarcoma can occur in any site on the body, but is primarily found in the head, neck, orbit, genitourinary tract, genitals, and extremities. Types of rhabdomyosarcoma include embryonal rhabdomyosarcoma, alveolar rhabdomyosarcoma, and anaplastic rhabdomyosarcoma.

Rhabdomyosarcoma can be difficult to diagnose due to its similarities to other cancers and varying levels of differentiation. It is loosely classified as one of the “small, round, blue-cell cancer of childhood” due to its appearance on an H&E stain. However, the defining diagnostic trait for rhabdomyosarcoma is confirmation of malignant skeletal muscle differentiation with myogenesis under light microscopy. Magnetic resonance imaging (MRI), ultrasonography, and a bone scan can be used to determine the extent of local invasion and metastasis.

Treatment of rhabdomyosarcoma is a multidisciplinary practice involving the use of surgery, chemotherapy, radiation, and possibly immunotherapy. Chemotherapy has been shown to be the most effective method for treating rhabdomyosarcoma. There are two main chemotherapeutic methods for the treatment of rhabdomyosarcoma. These are the VAC regimen, consisting of vincristine, actinomycin D, and cyclophosphamide, and the IVA regimen, consisting of ifosfamide, vincristine, and actinomycin D.

The present invention includes the use therapeutic targets identified by the present invention that contribute to indirect interference with PAX3-FOXO1 activity in rhabdomyosarcoma at the different molecular levels. Examples of therapeutic targets include upstream modifiers and activators, epigenetic and transcriptional co-regulators, and downstream effector targets. In some embodiments, the genomic region is at least a portion of a gene. The present invention includes a variety of methods of modulating the expression of a genomic region of a cancer cell. For examples of such methods, see Wachtel, M., and Schäfer, B., 2018. These methods can be used alone, or in combination with known methods of treating rhabdomyosarcoma such as chemotherapy. The expression of the genomic region is modulated (i.e., increased or decreased) by administering an effective amount of a nucleic acid. The nucleic acid may be included in a delivery system enabling efficient intracellular introduction. The delivery system may be preferably a vector, and both viral vector and non-viral vector may be used. The viral vector may include lentivirus, retrovirus, adenovirus, herpes virus and avipox virus vector, and the like may be used, but is not limited thereto.

In some embodiments of the method of treatment, the expression of the genomic region is decreased. Genetic methods such as the use of siRNA. ribozymes, or antisense RNA could also be used to suppress expression of a genomic region. For example, the expression can be decreased by administering an effective amount of a siRNA to the subject. siRNA is a duplex RNA which specifically cleaves target molecules to induce RNA interference (RNAi). Preferably, the siRNA of the present invention has a nucleotide sequence composed of a sense RNA strand homologous entirely or partially to a gene expressing a mutant NRF2 pathway protein nucleic acid sequence and an antisense RNA strand complementary thereto, which hybridizes with its target sequence within cells.

In other embodiments of the method of treatment, the expression of the genomic region is increased. For example, administering an effective amount of nucleic acids with sequences corresponding to mRNA or that active promoters can be used to increase the expression of a genomic region.

An example has been included to more clearly describe a particular embodiment of the invention and its associated cost and operational advantages. However, there are a wide variety of other embodiments within the scope of the present invention, which should not be limited to the particular example provided herein.

### EXAMPLE

### Example 1: Pioneer Activity of an Oncogenic Fusion Transcription Factor at Inaccessible Chromatin

In the present study, we address the possible retention of PF activity in the PAX3-FOXO1 fusion oncoprotein at the chromatin level, as a basis for understanding its role in FP-RMS initiation. Well-established features of bona fide PFs guide our investigation: (1) binding to repressed/compact/inaccessible chromatin; (2) nucleosomal motif recognition and occupancy. Our biochemical and high-resolution genomic analyses reveal steady-state association of PAX3-FOXO1 with repressed chromatin features, whereas kinetic studies of PAX3-FOXO1 induction reveal rapid targeting of PAX3-FOXO1 to nucleosome-occupied regions where the fusion is retained often without inducing accessibility. These findings reveal an interplay between PAX3-FOXO1 and H3K9me3 domain patterning, opening new avenues for further understanding the chromatin level role of PAX3-FOXO1 in heritable transmission of oncogenic events in FP-RMS.

### Results

We asked whether the fused PFs in rhabdomyosarcoma exhibited fundamental properties of known pioneers. Homology analysis of PAX3 and FOXO1 amino acid sequences revealed that evolutionarily conserved residues within the PAX3 paired domain and homeobox domain are completely retained in the fusion TF (FIG. 2A). While the conserved nuclear-export and -localization sequences (NES and NLS), as well as the transactivation domain (TAD) of FOXO1 remain intact in the fusion, the forkhead or winged-helix domain is N-terminally truncated. The PAX3-FOXO1 fusion product retains the essential amino acid residues within alpha-helix 3 for recognition of FOXO1 DNA sequence motifs (5′-TGTTTAC-3′) and the flexible wings W1 and W2, which have been shown to stabilize FOXO1:DNA interactions through direct phosphate-backbone contacts (FIG. 2A) (Brent et al., 2008). Despite the truncation, it was predicted by analysis of the primary amino acid sequence that the ordered DNA-binding domains and the disordered transactivation domains, originating from both PAX3 and FOXO1, are retained in the resulting fusion product. As the PAX3-FOXO1 chimera retains many of the conserved features of these pioneer protein families, we went on to systematically assess the ability of this fusion TF to initiate reprogramming at the chromatin level to better understand epigenetic initiation in FP-RMS.

We were first motivated to address the enigmatic question of how a driver oncogene like PAX3-FOXO1 can initiate chromatin reprogramming, as it has only been previously observed to bind active, accessible chromatin. Thus, we tested whether PAX3-FOXO1 genomic localization is distinct from non-pioneer TFs and whether it has the capacity to bind regions with lower levels of accessibility. We conducted a genome-wide correlation analysis between various chromatin factors and PAX3-FOXO1 using chromatin immunoprecipitation sequencing (ChIP-seq) datasets from the FP-RMS cell line RH4, available publicly or generated for this study (Cao et al., 2010; Gryder et al., 2017). Our results revealed remarkable dissimilarity between PAX3-FOXO1 binding and localization of FP-RMS core regulatory TFs (CRTFs; MYCN, MYOG, MYOD1), chromatin structural components (CTCF, RAD21), enhancer binding/regulatory factors (MED1, BRD4, p300), active histone modifications (H3K27ac, H3K9ac, H3K4me1), accessibility (ATACseq), and SWI/SNF chromatin remodeling complex subunits (BRD9, DPF2). Remarkably, genome-wide PAX3-FOXO1 signal showed the second strongest correlation with the heterochromatic histone modification, H3K9me3, behind the repressive H3K27me3 modification. This observation revealed a unique genomic binding preference for PAX3-FOXO1 compared with other CRTFs in FP-RMS cells and suggested PAX3-FOXO1 may substantially reside in inactive regions of the genome.

To determine whether the pattern of PAX3-FOXO1 occupancy we observed could be attributed to its localization outside the active, euchromatic nuclear compartment, we employed a stringent, sequential fractionation protocol. We aimed at distinguishing cellular components associated with increasingly insoluble compartments of the nucleus in a panel of RMS cells, including PAX3-FOXO1 fusion-negative cell lines (RD, SMS-CTR) and fusion-positive cell lines (RH4, RH30). We found that the euchromatic SWI/SNF subunit BAF155 and the CRTF, MYCN, were readily extracted from the chromatin fiber in all cells when exposed to 500 mM NaCl extraction buffer (soluble nucleus; FIG. 2B). Residual chromatin-bound BAF155 was almost exclusively observed in the sonication-sensitive chromatin fraction (soluble chromatin; FIG. 2B). Consistent with previous characterization of this euchromatic fraction (Becker et al., 2017), we found that it was largely depleted of constitutive heterochromatin components such as HP1a as well as repression and compaction-related histone modifications such as H3K9me3 and H4K20me1 (Becker et al., 2017). TATA binding protein (TBP), with its dual role in active gene transcription and mitotic bookmarking was readily observed in all nuclear and chromatin fractions including the residual insoluble chromatin pellet (FIG. 2B) (Teves et al., 2018). On blotting with an antibody recognizing the C terminus of FOXO1 (epitope retained in the PAX3-FOXO1 fusion), we observed nuclear staining for wild-type FOXO1 in RD and SMS-CTR cells that was largely excluded from the insoluble chromatin fraction. Unexpectedly, nuclear PAX3-FOXO1 in RH4 and RH30 cells was readily observed in the sonication-resistant, insoluble portion of the chromatin (FIG. 2B). Thus, the PAX3-FOXO1 fusion protein appears capable of invading compact, repressed chromatin. This behavior, necessary for PF-mediated cell fate transition, is an unexplored feature of this oncogenic factor, previously unknown in this tumor.

To further delineate the unique chromatin binding features of PAX3-FOXO1 in FP-RMS, we next sought to define its genome-wide occupancy profile by optimizing high-specificity, spike-in normalized ChIP-seq conditions in RH4 cells using a C-terminal FOXO1 antibody. Our method, which we have called per-cell ChIP-seq (pc-ChIP-seq), addresses global changes in ChIP signal resulting from differential chromatin content or output between cell lines and treatment conditions, by introducing known ratios of mouse spike-in cells prior to sonication (Gryder et al., 2020). We justified this methodology by analyzing input sequencing libraries to reveal vastly different relative genome sizes across RMS cell lines (range, 5.25-10.02 Gb). Upon sequencing, we benchmarked our data against previously published PAX3-FOXO1 ChIP-seq generated with a non-commercial antibody, pFM2 (epitope spanning the fusion breakpoint) (Cao et al., 2010). We found that our ChIP-seq conditions identified 69% of reported binding events, and with improved signal, we revealed 7,341 additional high-strength PAX3-FOXO1 sites. Employing identical ChIP conditions in additional RMS cell lines, integrated analysis of our spike-in normalized anti-FOXO1 ChIP-seq revealed reproducible binding profiles that were concordant between FP-RMS cell lines (FIG. 2C). In addition, although fusion-negative RMS cells (FN-RMS), particularly SMS-CTR, express nuclear FOXO1 (FIG. 2B), anti-FOXO1 ChIP-seq in these cells did not result in high confidence peak assignments (9 and 0 peaks called in RD and SMSCTR, respectively), demonstrating that our approach is highly specific for generating PAX3-FOXO1 ChIP-seq even in the presence of FOXO1. As an internal control of FOXO1 antibody specificity in our RH4 PAX3-FOXO1 ChIP-seq dataset, we also analyzed reads aligning to the mouse genome, detecting just 135 low-confidence peaks in C2C12 cells with a greater than 14-fold reduction in the Fraction of Reads in Peaks (FRiP) score relative to RH4 cells. By comparing our PAX3-FOXO1 data in FP-RMS cells against nearly 26,000 publicly available ChIP-seq datasets from human tissues and cell lines using the Cistrome DB Toolkit (Mei et al., 2017; Zheng et al., 2019), we further supported the specificity of our approach, finding no significant correlation between PAX3-FOXO1 binding and any public wild-type PAX3 or FOXO1 ChIPseq data. Taken together, our robust method of PAX3-FOXO1 genomic localization provides a quantitative measure of fusion protein binding across individual cell models, while suggesting distinct binding patterns for PAX3-FOXO1 relative to its wild-type constituents.

We further investigated the quality and specificity of our PAX3-FOXO1 ChIP-seq by performing motif analysis. Among known motifs curated by HOMER, a PAX3:FKHR motif derived from previously published PAX3-FOXO1 ChlPseq was the top enriched sequence (p-value=101625) (FIG. 2D) (Cao et al., 2010; Gryder et al., 2017). By convention, FKHR is synonymous with FOXO1, and we have used the “PAX3:FKHR” nomenclature throughout this study when referring to this motif from HOMER. De novo motif identification also returned a sequence with the highest similarity to the known PAX3:FKHR motif as the most enriched (p value=10-1753). This motif comprises a paired-domain and a homeobox recognition sequence arranged in a divergent head-to-head orientation separated by a single cytosine. The remaining motifs enriched within PAX3-FOXO1 peaks (RH4 and RH30) were mainly E-Box motifs recognized by the non-pioneer (Fernandez Garcia et al., 2019), extended alpha-helical DBDs of basic helix-loop-helix (bHLH) family TFs such as MYF5, MYOG, MYOD1, and TCF, many of which are expressed in FP-RMS cells (FIG. 2D). Of note, forkhead motifs were only modestly enriched in PAX3-FOXO1-binding sites relative to background (FOXO1 motif p value=10⁻³¹), suggesting that sequence-specific binding of the fusion is mediated predominantly through the intact PAX3 DBD. This was further supported by the existence of a transposase-protected footprint centered on high-confidence PAX3:FKHR motifs in RH4-binding sites, whereas no clear footprint was observed over forkhead motif positions by ATAC-seq (FIG. 2E). We proceeded with high confidence in our PAX3-FOXO1 (P3F) dataset as a foundation for discovering comprehensive genomic binding patterns and potential pioneer function of this oncogenic fusion protein.

Having identified thousands of novel P3F binding sites in the FP-RMS genome, we were motivated to understand the genomic context and characteristic epigenetic state at these regions. As previously described, we found that P3F mainly occupies non-promoter, predominantly distal intergenic regions. Linking each P3F peak to nearby transcription start sites (TSS) with GREAT, we found that the limited number of promoter and promoter-proximal P3F binding sites were associated with general cellular and metabolic pathways. Interestingly, more distal P3F binding sites up to 500 kilobases from a TSS were strongly linked to genes with neurogenic differentiation processes However, these neurogenic genes show similarly high expression across FP-RMS, FN-RMS, neuroblastoma, and glioma cell lines relative to all other models in the Cancer Cell Line Encyclopedia (CCLE). Thus, although our discovery of new P3F-binding sites reveals associations of this fusion oncoprotein with gene pathways beyond the myogenic transcriptional circuitry, we find that P3F expression and binding alone cannot predict high expression of these genes. Further investigation of the functional consequences of P3F chromatin binding may reveal an order of events, providing clues regarding a cell of origin.

As in previous studies (Cao et al., 2010; Gryder et al., 2017, 2019), we confirmed that P3F shows enriched occupancy of gene-regulatory enhancers compared with promoters, including 1,520 individual binding sites within 932 TSS-distal, high-intensity H3K27ac clusters stitched together by ROSE (FIG. 2F). However, we observed no correlation between H3K27ac strength and P3F occupancy in these sites. Clustered enhancers lacking the fusion oncoprotein exhibited nearly equal levels of H3K27ac compared with those bound by P3F (FIG. 2G). One explanation for these observations is that P3F occupies a narrow footprint within discrete enhancer elements and thus does not broadly influence the entire landscape of these broad regions (FIG. 2G). However, with a general lack of correlation between P3F binding and H3K27ac enrichment across all enhancers, our results are consistent with a limited role for P3F in enhancer acetylation. We found that 2,565 P3F sites occur outside of enhancers and confirmed that these regions are in fact depleted of both H3K27ac and H3K4me1 while maintaining DNA accessibility (FIG. 2F). Despite evolutionary conservation of the DNA sequences within these P3F sites, we could not readily discern their regulatory role within the FP-RMS epigenome using publicly available datasets. This suggests that these non-enhancer P3F peaks exhibit lineage-restricted epigenetic signatures, a feature common to promoter-distal gene-regulatory elements. To understand if these regions might be better classified as “poised,” we mapped our H3K27me3 ChIP-seq data to PAX3-FOXO1-binding sites compared with true H3K27me3-marked regions. Given the relative lack of H3K27me3 signal, combined with depletion of both H3K27ac and H3K4me1 at these P3F-bound sites, our data does not support classifying these regions as enhancers on the basis of their epigenetic signatures.

We next evaluated expression changes of genes linked to each type of P3F site, including non-enhancer regions. Through integrative meta-analyses of our P3F-bound sites with publicly available gene expression data, we did not observe substantial changes in the expression of genes proximal to P3F-binding sites associated with altered P3F expression (Gryder et al., 2017). This result was robust across conditions of P3F knockdown or add-back, as well as when comparing FP-RMS versus FN-RMS cell lines. On subsequent analyses of RNA sequencing (RNA-seq) profiles from a cohort of PAX3-FOXO1 +FP-RMS versus FN-RMS (embryonal, ERMS, subtype) patients (Downing et al., 2012), we once again found that relatively few genes associated with PAX3-FOXO1 binding were differentially expressed in patients based on PAX3-FOXO1 fusion status. Across P3Fbinding site categories, less than 30% of proximal genes were differentially expressed in patient samples (average 23.7%, fold change >2, adjusted p value <0.05), and these genes showed similar likelihood of being up- or down-regulated in P3F+ patients. These data indicate that P3F binding alone may be a poor predictor of gene activation in FP-RMS tumors and model systems, suggesting that additional inputs are required to initiate gene expression reprogramming in a context-dependent manner

We then asked whether the genomic occupancy profile of P3F related to our finding that the fusion oncoprotein is readily localized to the insoluble chromatin pellet in cell fractionation assays (FIG. 2B). To this end, we performed H3K9me3 ChIP and modified our ChIP-seq library preparation protocol to achieve better sequencing coverage across relatively sonication-resistant portions of the genome (FIG. 2H) (Becker et al., 2017). Aligning our resulting data to P3F-binding site categories, we found that H3K9me3 signals were specifically depleted from enhancer, clustered enhancer, and promoter binding sites, whereas non-enhancer sites were often associated with a local H3K9me3 peak directly overlapping P3F binding events (FIG. 2H). We noted a relatively high H3K9me3 signal flanking all P3F peaks, regardless of binding site category. We assessed whether these were balanced (both up- and downstream) versus asymmetric (only up- or downstream) H3K9me3 signals adjacent to P3F sites. We isolated H3K9me3 signals upstream of P3F sites from H3K9m3 signals downstream of P3F sites and ranked sites in each category according to their 50-30 imbalance. From these analyses, it was clear that the majority of P3F sites exhibit some degree of asymmetry in the adjacent H3K9me3 signal, and this occurs at each binding site category (FIG. 2H). Of importance, we found that enhancers generally did not display a strong up- or downstream H3K9me3 signal. In the rare occurrences that enhancers were flanked by strong, asymmetric H3K9me3 signals (6.8% of enhancers), these regions displayed relatively strong P3F occupancy (FIG. 21). This analysis of H3K9me3 brings clarity to our earlier genome-wide correlation analysis, which showed only moderate correlation between P3F and H3K9me3 signals, perhaps a result of P3F most often residing adjacent to a strong H3K9me3 signal with less frequent, direct overlap. In addition, we learn that the enrichment of P3F to the insoluble chromatin fraction of the nucleus may reflect its occupancy at the boundaries of H3K9me3 domains, corresponding to a rare subset of active regulatory elements immediately flanked by repressed chromatin. Together, the above results are suggestive of a pioneer role for PAX3-FOXO1 whose binding is associated with chromatin accessibility often on the edges of repressed chromatin tracts, but that may be insufficient for chromatin activation and highly context dependent for gene regulation.

### Early Genomic Targeting of PAX3-FOXO1 Exhibits Nucleosomal Motif Recognition

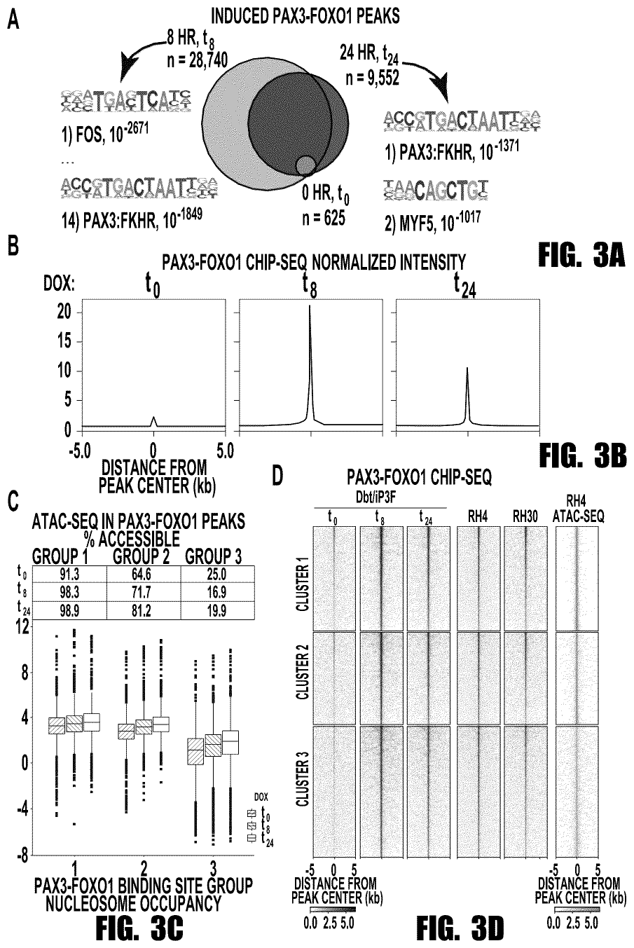

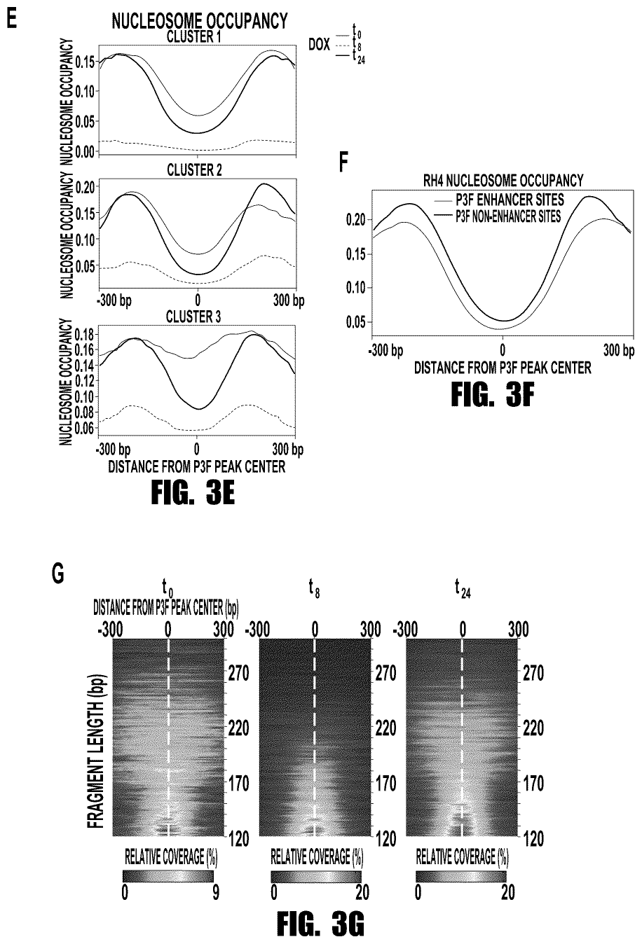

Although binding to inactive chromatin is a feature differentiating PFs from traditional TFs, another rigorous definition of nucleosomal motif binding may be applied to understand how a TF with transforming potential is capable of invading repressed sites (Fernandez Garcia et al., 2019). However, this function cannot be fully addressed at equilibrium, in which pioneer binding may in certain cases result in rapid nucleosome destabilization or eviction upon recruitment of additional factors (Yan et al., 2018). Observing this phenomenon requires kinetic regulation of PAX3-FOXO1 to monitor chromatin state changes over short timescales. To address the limitations of studying FP-RMS cells at steady state, we employed a model system of immortalized human myoblasts (Dbt), engineered with a doxycycline-inducible PAX3-FOXO1 construct (Dbt/iP3F) (Pandey et al., 2017). With kinetic control of P3F expression, we set out to establish the immediate-early targets of P3F binding and assess its capacity to recognize and occupy inaccessible, nucleosomal motifs. We first performed spike-in normalized P3F ChIP-seq in Dbt/iP3F cells with 0 (t0), 8 (t8), and 24 (t24) hours of doxycycline treatment. At t8, we identified 28,740 high-confidence P3F binding sites enriched primarily with bZIP and bHLH motifs, whereas the PAX3:FKHR motif ranked 14^{th} among overrepresented sequences (FIG. 3A). Interestingly, short bZIP DNA motifs share a high degree of sequence similarity with the central five nucleotides of the extended PAX3-FKHR motif in HOMER, suggesting that this initial phase of P3F binding involves frequent sampling of partial motifs. By t24, as P3F protein expression plateaus, the number of P3F sites was reduced to 9,552, with the PAX3:FKHR motif ranked first among enriched sequences (FIG. 3A). In addition to P3F-binding sites being less numerous at t24 compared with t8, sites at t24 also had a lower average intensity (FIGS. 3B). These results may be consistent with the reported slow mobility of pioneer factors throughout chromatin as they sample non-specific nucleosomal sites before equilibration in a DNA sequence-driven manner

ATAC-seq suggested that P3F sites in Dbt/iP3F cells universally exhibited increases in accessibility over the doxycycline treatment period, although we noted that many induced P3F sites begin with relatively high accessibility at t0. To understand if accessibility changes were limited to sites with low versus high initial accessibility, we distinguished P3F-binding sites in Dbt/iP3F cells according to their baseline accessibility signal. We defined P3F-binding site Groups 1, 2, and 3 with high, medium, and low initial accessibility at t0, respectively (FIG. 3C). In each Group, accessibility appeared to increase with time of P3F induction, although Group 3 sites showed lower ATAC-seq signal at t24 than either Group 1 or 2 sites at t0 (FIG. 3C). In fact, a relatively small percentage of Group 3 sites are defined as ATAC-seq peaks at t0, and few additional ATAC peaks overlapped Group 3 sites at t24. We refined Group 1, 2, and 3 regions to include only P3F sites that are observed at equilibrium in RH4 cells (referred to as Clusters 1, 2, and 3). ChlPseq profiles in these regions indicate that the high P3F occupancy observed at t8 in Dbt/iP3F cells is a transient state prior to the cells approaching equilibrium at t24, more closely resembling the binding profiles observed in FP-RMS cells (FIG. 3D). We observed that the accessibility differences between Clusters 1, 2, and 3 in Dbt/iP3F cells were clearly visible in RH4 ATAC-seq data, revealing that the Dbt/iP3F model faithfully recapitulates the targeting and retention of P3F to relatively inaccessible regions (FIG. 3D). Consistent with PF behavior reported previously, PAX3-FOXO1 binding in Cluster 3 sites infrequently overlapped with ATAC-seq peaks at t0 or with induced ATAC-seq peaks at t24. Overall, these patterns reveal that PAX3-FOXO1 rapidly targets to inaccessible chromatin regions, but the fusion protein is insufficient to induce accessibility at all binding locations. Differences in PAX3-FOXO1 signal intensity between 8- and 24-h timepoints may reflect a late equilibrium period of P3F chromatin binding and release as additional factors are recruited to these regions.

Finally, we applied the NucleoATAC pipeline to determine if nucleosome positioning is affected over the time course of P3F induction and genomic occupancy. At t0 we observed strong evidence for nucleosome occupancy in Cluster 1, 2, and 3 sites. Aligning nucleosome positions with respect to P3F peaks centers, we found that Cluster 1 and 2 regions, exhibiting clear accessibility, had evidence of a pre-established nucleosome-depleted region (NDR) flanked by up- and downstream nucleosomes prior to P3F induction (FIG. 3E). Cluster 3 sites with low baseline accessibility exhibited more uniform nucleosome occupancy prior to P3F induction, with less evidence of an NDR at t0. By t24 of P3F expression, the central NDR of Clusters 1 and 2 appeared to be further remodeled, as evidenced by a widening and deepening of the nucleosome occupancy curve. In addition, Cluster 3 sites showed evidence of de novo establishment of an NDR directly overlapping a portion of induced P3F binding events while evidence for well-positioned nucleosomes neighboring these P3F peaks remained. This provides evidence of P3F targeting nucleosome-occupied sites followed in some cases by rapid remodeling events in inaccessible regions of the genome. We observed nearly identical patterns of equilibrium nucleosome occupancy in RH4 cells, suggesting that extended periods of P3F expression may ultimately result in the recruitment of additional factors required for nucleosome remodeling at many P3F sites (FIG. 3F). We noted that NucleoATAC applied to our t8 ATAC-seq dataset was inefficient for inferring nucleosome positions in all Clusters (FIG. 3E). Considering the NucleoATAC model inputs, we hypothesized that this was the result of differences in nucleosome-length fragments mapping to P3F sites, where a relative depletion of longer nucleosomal versus shorter sub-nucleosomal fragments would result in low confidence nucleosome occupancy scores. Using plot2DO (Beati and Chereji, 2020), we in fact observed differences in fragment length distributions consistent with our NucleoATAC result (FIG. 3G). We interpret this anomaly in nucleosome length fragment distribution as evidence of local nucleosome redistribution taking place during the transition between baseline (t0) and P3F-driven epigenetic equilibrium (t24). Supporting the involvement of direct P3F binding in this nucleosome disruption event, we observed that Tn5 insertion rates generally increased over time in all P3F site Clusters, and particularly in Cluster 3, we observed the gradual formation of a Tn5-protected footprint centered on high-confidence PAX3:FKHR motifs at t8 and t24. Although NucleoATAC provides some evidence that nucleosome positioning is altered upon P3F binding to chromatin, more than 70% of Cluster 3 sites remain largely inaccessible even 24 h after P3F expression. This large subset of P3F binding events reveal that the fusion can establish a DNA occupancy footprint in regions otherwise inaccessible to traditional TFs and is likely retained in these sites as necessary for the recruitment of additional chromatin remodeling factors.

### Discussion

We have critically evaluated the PAX3-FOXO1 fusion oncoprotein with respect to categorical properties intrinsic to the pioneer class of transcription factors. Advances in recent years have catalyzed integrations across fields including pediatric oncology, genomics, and the biophysics of pioneer transcription factors (Fernandez Garcia et al., 2019; Nacev et al., 2020). Our studies have revealed that, for two pioneer factors fused as a chimeric oncoprotein in a rare childhood tumor, chromatin recognition is consistent with PF function across the genome, including steady-state association with inactive and H3K9me3-marked domains and kinetic recognition of nucleosomal motifs. In developing quantitative per-cell normalization for our genome-wide binding studies (pc-ChIP-seq; STAR Methods), we are able to infer nucleosome-targeting for PAX3-FOXO1, while accounting for sequencing bias resulting from the non-diploid genome structure of rhabdomyosarcoma models (Chen et al., 2015). We have demonstrated pioneer activity of the most common driver alteration in fusion-positive rhabdomyosarcoma (Galili et al., 1993), which had previously uncharacterized function outside of active chromatin (Cao et al., 2010; Gryder et al., 2017, 2019). Future efforts will be important to understand the kinetic rate constants of PAX3-FOXO1 dissociation from nucleosomes containing its motif, as well as focused chromatin sequencing of sonication-resistant binding sites within and adjacent to heterochromatin domains (Becker et al., 2017). These efforts will be necessary to understand the role of PAX3-FOXO1 in heritable transmission of FP-RMS phenotypes through the establishment and maintenance of stable epigenetic states. In the coming years we anticipate continued convergence of fields, with emerging evidence to understand and predict the logic of pioneer activity in development and disease.

### Materials and Methods

### Cell Lines

Cell lines used in this study include mouse C2C12 myoblasts (female), PAX3-FOXO1+ FP-RMS cells RH4 (human, female) and RH30 (human, male), FN-RMS cells SMS-CTR (human, male) and RD (human, female), and immortalized human myoblast cells Dbt and Dbt/iP3F (male).

### Cell Culture

RH4 (FP-RMS), RH30 (FP-RMS), RD (FN-RMS), and SMS-CTR (FN-RMS) cells, a gift from Dr. Peter Houghton (UTHSCSA), were cultured in high-glucose DMEM supplemented with 10% FBS, Glutamax, and penicillin/streptomycin Immortalized Dbt myoblasts engineered with doxycycline-inducible PAX3-FOXO1 (Dbt/iP3F), engineered in the lab of Dr. Frederic Barr (NIH/NCI) (1), were cultured in Ham' s/F-10 supplemented with 15% FBS, glutamine, sodium pyruvate, creatine monohydrate, uridine, and penicillin/streptomycin. Mouse C2C12 myoblasts were purchased from ATCC and cultured in high-glucose DMEM supplemented with 10% FBS, Glutamax, and penicillin/streptomycin. For PAX3-FOXO1 induction studies, Dbt/iP3F cells were seeded in normal culture media and grown to approximately 70% confluence before exchange with media containing 500 ng/mL doxycycline hyclate for the desired time period (8 or 24 hrs).

### Cell Fractionation and Immunoblotting

Cells were collected from 15 cm plates and washed with ice-cold PBS containing protease inhibitor cocktail. Cell pellets were resuspended in Fractionation Buffer 1 (20 mM HEPES, 10 mM KCl, 0.2 mM EDTA) with protease inhibitor cocktail and incubated for 10 minutes on ice. NP-40 was added to a final concentration of 0.5% and the samples was vortexed on high for 15 seconds. Samples were incubated on ice for 1 minute, vortexed at high speed for 15 seconds, and pelleted at 14,000 rpm for 1 minutes at 4° C. The supernatant (cytoplasmic fraction) was transferred to a clean Eppendorf tube, and the nuclei pellet was resuspended in Fractionation Buffer 2 (10 mM Tris-HCl pH 8.0, 1 mM EDTA, 0.1% NP-40, 500 mM NaCl) with protease inhibitor cocktail and incubated at 4° C. for 45-60 minutes with overhead rotation. The sample was centrifuged at 14,000 rpm for 10 minutes and the supernatant (soluble nuclear fraction) was transferred to a clean Eppendorf tube. The remaining chromatin pellet was resuspended in IP Buffer (50 mM Tris-HCl pH 8.0, 300 mM NaCl, 1 mM EDTA, 1% Triton X-100) with protease inhibitor cocktail and sonicated with an Active Motif EpiShear probe sonicator equipped with a cooled sonication platform for 5 minutes at 30% amplitude cycling from 30 seconds ON to 30 seconds OFF. The sample was centrifuged at 14,000 rpm for 10 minutes at 4° C. and the supernatant (soluble chromatin fraction) was transferred to a clean Eppendorf tube. The remaining chromatin pellet was resuspended in 2×SDS protein sample buffer with 10% v/v 2-mercaptoethanol and heated at 95° C. for 10 minutes. The other cell fractions were quantified with the Pierce Rapid Gold BCA Protein Assay Kit. Samples were resolved by SDS-PAGE using 2.5 μg of the cytoplasmic, soluble nuclear, and soluble chromatin fractions or 10 μL of the chromatin pellet fractions on a NuPAGE 4-12% Bis-Tris gel.

Proteins were transferred overnight at 4° C./30V to nitrocellulose membranes. Membranes were blocked at room temperature in 5% w/v milk solution in TBST (0.1% v/v Tween-20) for 1 hour before incubation for 2 hours at room temperature with primary antibodies detecting: MYCN (Cell Signaling, 51705S), BAF155 (Cell Signaling, 11956S), FOXO1 (Cell Signaling, 2880S), HP1a (Cell Signaling, 2616S), H3K9me3 (Active Motif, 39062), H4K20me1 (Active Motif, 39727), or TBP (Cell Signaling, 44059S). Following 1 hour incubation with HRP-linked secondary antibodies, immunoblots were incubated with SuperSignal West Pico PLUS Chemiluminescent Substrate, images were acquired with a LI-COR C-DiGit Blot Scanner running Image Studio v5.2.

### Amino Acid Primary Sequence Analysis

Human PAX3 (Uniprot ID: P23760) and FOXO1 (Uniprot ID: Q12778) amino acid sequence conservation was analyzed on the ConSurf Server using default parameters (Berezin et al., 2004). ConSurf output files were used to project evolutionary conservation estimates, buried/exposed residue classifiers, and functional/structural residue classifiers onto the primary protein structure in FIG. 2A. Protein disorder analysis was conducted using the VL-XT PONDR algorithm (Romero et al., 2001) on the wild type PAX3 and FOXO1 protein sequences as well as the PAX3-FOXO1 fusion protein sequence.

### PAX3-FOXO1 Spike-In ChIP Optimization

To facilitate quantitative normalization across ChIP-seq samples not only from different treatment conditions but also from distinct cell lines, we employed a spike-in ChIP strategy using known numbers of cells of human origin mixed in defined ratios with cells of mouse origin. This approach is similar in theory and in practice to the recently reported quantitative HiChIP method, known as AQuA-HiChIP (Gryder et al., 2020). Our spike-in strategy addresses technical confounders introduced at two key points in the ChIP-seq protocol. Firstly, we introduce formaldehyde fixed mouse C2C12 cells to fixed human RMS or myoblast cells prior to sonication. In this study, the ratio of humanmouse cells is fixed at 3:1 for all experiments. This strategy ensures subsequent normalization steps retain read depth information on the basis of starting cell number. Failure to do so may obscure differences in chromatin output per cell across different cell types and treatment conditions. The assumption of equal chromatin produced per cell by sonication of any cell type implicit in commercial spike-in normalization reagents may be frequently violated when conducting experiments comparing aneuploid cancer cell lines, or in our case, RMS cells in which genome duplication may be a frequent event. Secondly, as in previous ChIP-Rx and commercial spike-in strategies (Egan et al., 2016), we assume an equal number of mouse DNA fragments comprise the final sequencing libraries for input samples and for ChIP samples that will be directly compared. This permits us to properly correct for differences in sequencing depth across samples based on an internal and constant reference.

Our strategy utilizes one antibody, which we ideally expect to react with specific, conserved epitopes on human and mouse chromatin (e.g. histone modifications). In this ideal case, reliable and stable ChIP efficiency against epitopes on mouse chromatin ensures adequate and reproducible read numbers mapping to the spike-in mouse genome across all samples, while distinct human cell lines (e.g. RMS cells vs. myoblasts) under various treatment conditions (e.g. doxycycline induction) may produce a variable number of reads mapping to the human genome. In the case that an antibody has exquisite species-specific reactivity, or the desired epitope is not expressed in C2C12 cells (e.g. lineage restricted transcription factors), we leverage the inherently low signal-to-noise ratio of ChIP assays to our advantage. Here the number of non-specific reads mapping to the mouse genome is anticipated to remain constant across all ChIP samples, as even in highly efficient ChIP assays, background reads are present in relatively high proportions. Therefore, a single antibody approach is sufficient to produce ChIP samples with a constant number of spike-in reads mapping to the mouse genome for a given antibody, regardless of the reactivity of that antibody with mouse epitopes.

In addition to developing a novel, per-cell ChIP (pc-ChIP) approach, we tested two variables to optimize conditions for PAX3-FOXO1 ChIP using a FOXO1 antibody: 1) sonication time and 2) salt concentration in the ChIP buffer. The detailed pc-ChIP optimization strategy follows:

### Cell Fixation

FP-RMS, FN-RMS, Dbt/iP3F, or C2C12 cells were cultured in 15 cm plates, dissociated with trypsin, pelleted, and washed with PBS. Cell pellets were resuspended in Fixing Buffer (50 mM HEPES pH 7.3, 1 mM EDTA, 0.5 mM EDTA, 100 mM NaCl) and fresh, methanol-free formaldehyde was added to a final concentration of 1%. After 10 minutes of incubation at room temperature, the fixation was quenched by the addition of glycine to a final concentration of 125 mM and the cell suspension was placed on ice for 5 minutes. Fixed cells were pelleted at 1,200×g for 5 minutes at 4° C. and resuspended in ice-cold PBS containing a protease inhibitor cocktail. Fixed FP-RMS, FN-RMS, and Dbt/iP3F cells were aliquoted at 6×10⁶ cells/tube, and fixed C2C12 cells were aliquoted at 2×10⁶ cells/tube. Cells were pelleted at 3,000 rpm for 5 minutes at 4° C., the supernatant was removed, and pellets were snap frozen and stored at −80° C. until further use.

### Sonication

For initial optimization in RH4 cells, thawed aliquots of 6×10⁶ RH4 cells were combined with thawed aliquots of 2×10⁶ C2C12 cells in 800 uL of TE buffer pH 8.0 containing protease inhibitor cocktail and transferred to polystyrene sonication tubes. Samples were sonicated with an Active Motif EpiShear probe sonicator equipped with a cooled sonication platform for either 27 minutes or 13.5 minutes at 30% amplitude cycling from 30 seconds ON to 30 seconds OFF. After sonication, a 5 uL volume was aliquoted from each sample (input) and combined with 20 uL TE, 1 uL 10% SDS, and 1 uL 20 mg/mL Proteinase K for overnight decrosslinking at 65° C. The remaining sonicated chromatin was stored at 4° C. Input samples were purified using Qiagen MinElute PCR Purification columns and chromatin fragmentation was assessed by gel electrophoresis on an E-Gel 2% EX agarose gel.

### ChIP

After evaluating fragmentation, sonicated chromatin in TE buffer was adjusted to ChIP Buffer by the addition of Triton X-100 (to 1% final concentration), SDS (to 0.1% final concentration), and sodium deoxycholate (to 0.1% final concentration). For initial optimization, we added sodium chloride to a final concentration of either 140 mM or 200 mM. Chromatin in ChIP Buffer was incubated on ice for 5 minutes and insoluble material was removed by centrifuging the sample at 13,000 rpm for 10 minutes at 4° C. and transferring the supernatant to a new 1.5 mL tube. 5 uL of FOXO1 antibody (Cell Signaling Technology, #2880) was added, and samples were incubated at 4° C. for 1 hour with overhead rotation. For each sample, 40 uL of Protein A Dynabeads were buffer exchanged with ChIP buffer containing either 140 mM or 200 mM NaCl before addition to the antibody:chromatin mixture, and the samples were incubated overnight at 4° C. with overhead rotation. The beads were then washed twice with Low Salt Wash Buffer (0.1% SDS, 0.1% sodium deoxycholate, and 1% Triton X-100 in TE buffer pH 8.0), twice with ChIP Buffer (containing either 140 mM or 200 mM NaCl), twice with LiCl Wash Buffer (250 mM LiCl, 0.5% NP-40, 0.5% sodium deoxycholate in TE buffer pH 8.0), and twice with TE buffer pH 8.0. The beads were then resuspended in 100 uL TE buffer pH 8.0 with 2.5 uL 10% SDS and 5 uL 20 mg/mL Proteinase K, and ChIP samples were decrosslinked at 65° C. overnight. ChIP DNA was purified with Qiagen MinElute PCR Purification columns.

### Real-Time PCR Validation

Efficiency and specificity of our PAX3-FOXO1 ChIP conditions were first assessed by real-time PCR with primers designed against known PAX3-FOXO1 binding sites within the MYOD1, SOX8, QKI, RAD51B, and FGFR4 loci (primer sequences listed in Table 1 below) (Cao et al., 2010). A negative control region in the SOX18 promoter was also tested (Yohe et al.). This analysis revealed specific and robust ChIP enrichment within known PAX3-FOXO1 binding sites compared to the negative control region.

TABLE 1

| | Forward (5′-3′) | Reverse (5′-3′) | hg38 Region |
|---|---|---|---|
| MYOD1 | CAGAACCATCCCATTCTCCG | GCCTGACCTTGAACGTGAAT | chr11: |
| | (SEQ ID NO: 3) | (SEQ ID NO: 4) | 17650441-17650563 |
| SOX8 | GGATCGTGTAACCTGAGGGC | CAGTGAGTGTCTGCCTGCAA | chr16: |
| | (SEQ ID NO: 5) | (SEQ ID NO: 6) | 1042251-1042350 |
| QKI | TGCATGCTGGTGACAGATCA | ACAGCGTCCTCTTTCAGCTT | chr6: |
| | (SEQ ID NO: 7) | (SEQ ID NO: 8) | 163777913-163778066 |
| RAD51B | TTCCCATGAAAGGAGAAGCAGA | GGAAATGCCTCCACAGAAACG | chr14: |
| | (SEQ ID NO: 9) | (SEQ ID NO: 10) | 68552591-68552741 |
| FGFR4 | AAATTTGACCTTCGTCGGCAC | CAGCTGTTGGCGATTTCACG | chr5: |
| | (SEQ ID NO: 11) | (SEQ ID NO: 12) | 177105827-177105916 |
| SOX18 | GCTCTTGGTTCTCTGTCCCT | AGACAGACTGTGATGTGGGG | chr20: |
| | (SEQ ID NO: 13) | (SEQ ID NO: 14) | 64050790-64051013 |

This optimization strategy revealed that 13.5 minutes of sonication followed by ChIP in buffer containing 200 mM NaCl was suitable for robust PAX3-FOXO1 ChIP enrichment using a FOXO1 antibody with limited background signal. All subsequent PAX3-FOXO1 ChIP assays in RH4, RH30, RD, SMS-CTR, and Dbt/iP3F cells were performed in an identical fashion.

Additional ChIP assays performed for this study were conducted in RH4 cells (with C2C12 spike-in) sonicated for 27 minutes, with immunoprecipitation performed in ChIP buffer with 200 mM NaCl using antibodies against DPF2 (Abcam, ab134942), BRD9 (Abcam, ab137245), H3K27ac (Active Motif, 39133), and H3K27me3 (Active Motif, 39155). H3K9me3 (Active Motif, 39062) and H3K9ac (EpiCypher, 13-0020) ChIPs were performed in RH4 cells (with C2C12 spike-in) sonicated for 12 minutes.

### pc-ChIP-seq Considerations

We perform our pc-ChIP-seq spike-in and normalization on a “per cell” basis to retain information regarding the chromatin output across cell lines tested under various treatment conditions. Conversely, commercially available spike-in reagents recommend an equal starting amount of chromatin for each sample, regardless of starting cell number. For direct, quantitative comparison to be performed between distinct cell lines and treatments, the condition of equal chromatin produced per cell in all groups must be satisfied when normalizing on the basis of starting chromatin amount. In RMS cell lines and other aneuploid cancer cell lines, this condition is grossly violated. These cell lines generally contain variably sized genomes, and therefore they release different amounts of chromatin per cell that can globally influence the signal output from a standard ChIP assay. Using human/mouse read ratios in our input sequencing libraries, we can demonstrate the need to account for this by inferring the relative genome size for each cell line in this study. We used tetraploid C2C12 mouse cells for spike-in (estimated genome size, 5.4×10⁹ bp) at a 3 to 1 ratio of human to mouse cells. We can therefore calculate the inferred genome sizes as:

Inferred Genome Size=(Observed Ratio×5.4×10⁹)/3

TABLE 2

| Observed Read Ratio (human/mouse) | Inferred Genome Size (×10⁹ bp) |
|---|---|
| RH4 Input = 4.3 | 7.74 |
| RH30 Input = 2.92 | 5.26 |
| RD Input = 5.57 | 10.02 |
| SMS-CTR Input = 3.1 | 5.58 |
| Dbt/iP3F Input = 3.18 | 5.72 |

These figures reveal vastly different relative genome sizes across cell lines tested and showed consistent results across RH4 replicates (7.72 and 7.92×10⁹ bp, respectively). This is consistent with the scenario where chromatin output differs globally, and therefore relative read depth across both input and ChIP samples differ. Under these conditions, a spike-in and normalization procedure based on the starting amount of chromatin instead of the starting number of cells would obscure the differences between different biological samples. This prevents qualitative comparison of peak calls as wells as quantitative comparison of signal strength observed. Therefore, we needed a new strategy where the difference of amount of chromatin released by each cell is taken into consideration, and we developed pc-ChIP-seq to correct this bias. While the focus here is on chromosome ploidy resulting in differential chromatin output per cell, epigenetic repression and de-repression as well as cell cycle stage are examples of other conditions that may globally influence chromatin output per cell owing to differences in sonication sensitivity.

To justify the assumption that a one-antibody strategy is sufficient for a spike-in ChIP-seq normalization approach, even in the event that the chosen antibody does not recognize epitopes on mouse chromatin to produce high quality ChIP-seq data in the spike-in genome, we first analyzed our anti-FOXO1 ChIP-seq data from RH4/C2C12 cells with the ENCODE pipeline, aligning only to mm10. This analysis, prior to down-sampling, revealed just 135 FOXO1 peaks in the mouse genome. More importantly, just 0.18% of all reads aligning to the mouse genome mapped to these peaks. Second, in replicate PAX3-FOXO1 ChlPseq datasets produced in RH4 cells, the ratio of human to mouse reads in the ChIP samples were 4.57 and 4.52. Together, these findings suggest even when non-specific mouse reads are not informative for peak calling in the mouse genome, they comprise a substantial and reproducible portion of a ChIP sample that can be leveraged for normalization. This reflects the inherently low signal-to-noise ratio of ChIP assays, where a typical transcription factor ChIP will often have a relatively low Fraction of Reads in Peaks (FRiP) value.

### pc-ChIP Library Construction and Sequencing

ChIP and input DNA from each sample were prepared for sequencing as before (Kidder and Zhao, 2014) by blunt end repair using the Lucigen End-It DNA End-Repair Kit, 3′ A-tailing by Klenow fragment (3′-5′ exo-), adaptor ligation by T4 DNA ligase, and size selection on an E-Gel 2% EX agarose gel. Libraries were amplified with barcoded primers for 14 cycles and isolated from unreacted primers by gel purification. Pooled libraries were sequenced at the Nationwide Children's Hospital Institute for Genomic Medicine, Genomic Services Laboratory on a HiSeq4000 running in paired-end, 150 bp mode.

### pc-ChIP-seq Normalization

As the pc-ChIP protocol rigorously controls for the starting number of cells and the ratio of human to mouse cells across all samples in each experimental group, read depth bias correction is achieved through normalization using mouse spike-in reads across all input and ChIP samples for a given comparison. We carried out normalization prior to peak calling and generation of signal files in order to perform quantitative comparisons across cell lines or test samples. In detail, our approach for random down-sampling of sequencing reads across samples is as follows:

BCL converted paired end fastq files were aligned to hg38 and mm10 reference genomes, separately, utilizing bowtie2 (Langmead and Salzberg, 2012) and following ENCODE best practices. The resulting mouse BAMs were analyzed by samtools to calculate the number of reads aligned to each genome. The minimum number of mouse aligned reads (m) was identified across all samples, and was divided by the number of aligned mouse reads for each sample(s) to calculate the scaling factor (f), f=m/s. This scaling factor was subsequently used to normalize the hg38 aligned BAMs through subsampling with samtools view -s, which retains read pair information. Picard SamToFastq converted the resulting SAM files to paired end fastq files.

### ChIP-seq Data Analysis

Normalized pc-ChIP-seq fastq files from this study as well as publicly available PAX3-FOXO1 ChIP-seq fastq files for RH4 cells (GSE19063, (Cao et al., 2010)) were processed with the ENCODE ChIP-seq pipeline with chip.xcor_exclusion_range_max set at 30. Normalized, paired-end fastqs were aligned with bowtie2 (version 2.3.4.3) to hg38, with parameters bowtie2-X2000-mm. Next, blacklisted region, unmapped, mate unmapped, not primary alignment, multi-mapped, low mapping quality (MAPQ<30), duplicate reads and PCR duplicates were removed. Peaks were called with MACS2 (version 2.2.4), with parameters-p 1e-2-nomodel-shift 0-extsize $[FRAGLEN]-keep-dup all-B-SPMR, where FRAGLEN is the estimated fragment length. IDR analyses were performed on peaks from replicate samples or pseudo-replicates, with threshold 0.05. Motif analysis with HOMER (version 4.11.1) (Heinz et al., 2010) was then carried out on conservative IDR peaks. For visualization, bedGraph files were generated with MACS2 bdgcmp from the pile-up, and then converted to bigwig format with bedGraphToBigWig. Heatmaps were generated with deeptools (version 3.3.1). k-means classification from deeptools was used to generate peak clusters for FIG. 3 ROSE (v0.1) (Whyte et al., 2013) was used with our H3K27ac IDR peaks to classify enhancers and to stitch proximate H3K27ac regions into clustered enhancers.

### ATAC-seq and ATAC-seq Data Analysis

ATAC-seq was performed as previously described (Buenrostro et al., 2013) with only minor modifications. 5×10⁴ cells per experiment were first washed with RSB buffer (10 mM Tris-HCl pH 8, 10 mM NaCl, 3 mM MgCl₂) and gently permeabilized with RSB lysis buffer (10 mM Tris-HCl pH 8, 10 mM NaCl, 3 mM MgCl₂, 0.1% NP-40) on ice. Cells were suspended in 50 uL of tagmentation master mix prepared from Illumina Tagment DNA TDE1 Enzyme and Buffer Kit components (#20034197), and transposition was performed for 30 minutes at 37° C. Tagmented DNA fragments were isolated using Qiagen MinElute PCR Purification columns prior to library amplification. ATAC-seq libraries were amplified with barcoded Nextera primers for 14 cycles, and excess primers were removed by size selection with AMPure XP beads. Libraries were sequenced on the HiSeq4000 platform running in PEx150bp mode.

The ENCODE ATAC-seq pipeline with default parameters was used to process ATAC-seq data. First, reads are scanned for adaptor sequences and trimmed with cutadapt (version 2.3). Reads are then mapped to hg38 with bowtie2 (version 2.3.4.3). Properly aligned, non-mitochondrial read pairs were retained for peak calling with MACS2 (version 2.2.4). After peaks are called, heatmaps are generated with deeptools (version 3.3.1) (Ramierez et al., 2016). Fragment length distributions were generated with plot2DO v1.0 (Beati and Chereji, 2020). Local signal vs background enrichment is calculated with localEnrichmentBed. Nucleosome position, nucleosome occupancy and tn5 insertion density are estimated using NucleoATAC (version 0.3.4) (Schep et al., 2015). Transcription factor foot printing/motif protection was assessed by identifying PAX3-FKHR or FOXO1 motif positions within PAX3-FOXO1 binding sites using FIMO (Grant et al., 2011). Insertion rates were subsequently plotted over aligned motif positions using deeptools (version 3.3.1).

### RNA-seq Analysis

Tumor tissue expression data (in bam format) from pediatric patients diagnosed with Alveolar Rhabdomyosarcoma (ARMS) and Embryonal Rhabdomyosarcoma (ERMS) were obtained from the St. Jude Cloud Genomics Platform (Downing et al., 2012). Reads were converted to gene level expression using featureCount (Rsubread package, version 2.4.3) with the Rsubread package built-in annotation (NCBI RefSeq annotation for hg38, build 38.2). Differential expression analyses were carried out between ARMS with PAX3-FOXO1 fusion biomarker (n=21) and ERMS (n=43) using DESeq2 (version 1.28.1). All analyses done in R version 4.0.0.

### LISTING OF REFERENCES

Arndt et al., (2009). Vincristine, actinomycin, and cyclophosphamide compared with vincristine, actinomycin, and cyclophosphamide alternating with vincristine, topotecan, and cyclophosphamide for intermediate-risk rhabdomyosarcoma: children's oncology group study D9803. J Clin Oncol, 27(31):5182-8.
Beati, P., and Chereji, R. V. (2020). Creating 2D occupancy plots using plot2DO. Methods Mol. Biol. 2117, 93-108.
Becker, J. S., McCarthy, R. L., Sidoli, S., Donahue, G., Kaeding, K. E., He, Z., Lin, S., Garcia, B. A., and Zaret, K. S. (2017). Genomic and proteomic resolution of heterochromatin and its restriction of alternate fate genes. Mol. Cell 68, 1023-1037.e15.
Berezin, C., Glaser, F., Rosenberg, J., Paz, I., Pupko, T., Fariselli, P., Casadio, R., and Ben-Tal, N. (2004). ConSeq: the identification of functionally and structurally important residues in protein sequences. Bioinformatics 20, 1322-1324.
Birrane, G., Soni, A., and Ladias, J. A. (2009). Structural basis for DNA recognition by the human PAX3 homeodomain. Biochemistry 48, 1148-1155.
Brent, M. M., Anand, R., and Marmorstein, R. (2008). Structural basis for DNA recognition by FoxO1 and its regulation by posttranslational modification. Structure 16, 1407-1416.
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA binding proteins and nucleosome position. Nat. Methods 10, 1213-1218.
Bulut-Karslioglu, A., Perrera, V., Scaranaro, M., de la Rosa-Velazquez, I. A., van de Nobelen, S., Shukeir, N., Popow, J., Gerle, B., Opravil, S., Pagani, M., et al. (2012). A transcription factor based mechanism for mouse heterochromatin formation. Nat. Struct. Mol. Biol. 19, 1023-1030.
Cao, L., Yu, Y., Bilke, S., Walker, R. L., Mayeenuddin, L. H., Azorsa, D. O., Yang, F., Pineda, M., Heiman, L. J., and Meltzer, P. S. (2010). Genome-wide identification of PAX3-FKHR binding sites in rhabdomyosarcoma reveals candidate target genes important for development and cancer. Cancer Res. 70, 6497-6508.
Chen, L., Shern, J. F., Wei, J. S., Yohe, M. E., Song, Y. K., Hurd, L., Liao, H., Catchpoole, D., Skapek, S. X., Barr, F. G., et al. (2015). Clonality and evolutionary history of rhabdomyosarcoma. PLoS Genet. 11, e1005075.
Cirillo, L. A., Lin, F. R., Cuesta, I., Friedman, D., Jarnik, M., and Zaret, K. S. (2002). Opening of compacted chromatin by early developmental transcription factors HNF3 (FoxA) and GATA-4. Mol. Cell 9, 279-289.
Cirillo, L. A., McPherson, C. E., Bossard, P., Stevens, K., Cherian, S., Shim, E. Y., Clark, K. L., Burley, S. K., and Zaret, K. S. (1998). Binding of the winged-helix transcription factor HNF3 to a linker histone site on the nucleosome. EMBO J. 17, 244-254.
Deluz, C., Friman, E. T., Strebinger, D., Benke, A., Raccaud, M., Callegari, A., Leleu, M., Manley, S., and Suter, D. M. (2016). A role for mitotic bookmarking of SOX2 in pluripotency and differentiation. Genes Dev. 30, 2538-2550.
Deng et al., (2012). FoxO1 inhibits sterol regulatory element-binding protein-1c (SREBP-1c) gene expression via transcription factors Sp1 and SREBP-1c. J Biol Chem., 287:20132-43.
Downing, J. R., Wilson, R. K., Zhang, J., Mardis, E. R., Pui, C. H., Ding, L., Ley, T. J., and Evans, W. E. (2012). The pediatric cancer genome project. Nat. Genet. 44, 619-622.
Egan, B., Yuan, C. C., Craske, M. L., Labhart, P., Guler, G. D., Arnott, D., Maile, T. M., Busby, J., Henry, C., Kelly, T. K., et al. (2016). An alternative approach to ChIP-seq normalization enables detection of genome-wide changes in histone H3 lysine 27 trimethylation upon EZH2 inhibition. PLoS One 11, e0166438.
Fernandez Garcia, M., Moore, C. D., Schulz, K. N., Alberto, O., Donague, G., Harrison, M. M., Zhu, H., and Zaret, K. S. (2019). Structural features of transcription factors associating with nucleosome binding. Mol. Cell 75, 921-932.e6.
Galili, N., Davis, R. J., Fredericks, W. J., Mukhopadhyay, S., Rauscher, F. J., 3rd, Emanuel, B. S., Rovera, G., and Barr, F. G. (1993). Fusion of a fork head domain gene to PAX3 in the solid tumour alveolar rhabdomyosarcoma. Nat. Genet. 5, 230-235.
Grant, C. E., Bailey, T. L., and Noble, W. S. (2011). FIMO: scanning for occurrences of a given motif. Bioinformatics 27, 1017-1018.
Gryder, B. E., Khan, J., and Stanton, B. Z. (2020). Measurement of differential chromatin interactions with absolute quantification of architecture (AQuA-HiChIP). Nat. Protoc. 15, 1209-1236.
Gryder, B. E., Pomella, S., Sayers, C., Wu, X. S., Song, Y., Chiarella, A. M., Bagchi, S., Chou, H. C., Sinniah, R. S., Walton, A., et al. (2019). Histone hyperacetylation disrupts core gene regulatory architecture in rhabdomyosarcoma. Nat. Genet. 51, 1714-1722.
Gryder, B. E., Yohe, M. E., Chou, H. C., Zhang, X., Song, Y., Gualtieri, A., et al. (2017). PAX3-FOXO1 establishes myogenic super enhancers and confers BET bromodomain vulnerability. Cancer Discov. 7, 884-899.
Hatta, M., and Cirillo, L. A. (2007). Chromatin opening and stable perturbation of core histone:DNA contacts by FoxO1. J. Biol. Chem. 282,35583-35593.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol. Cell 38,576-589.
Herman, L., Todeschini, A. L., and Veitia, R. A. (2021). Forkhead transcription factors in health and disease. Trends Genet. 37,460-475.
Hoffman. E., Frey, B., Smith, L., Auble, D., Formaldehyde crosslinking: a tool for the study of chromatin complexes. J Biol Chem (2015) 290(44):26404-11.
Iwafuchi-Doi, M., Donahue, G., Kakumanu, A., Watts, J. A., Mahony, S., Pugh, B. F., Lee, D., Kaestner, K. H., and Zaret, K. S. (2016). The pioneer transcription factor FoxA maintains an accessible nucleosome configuration at enhancers for tissue-specific gene activation. Mol. Cell 62,79-91.
Kadoch, C., and Crabtree, G. R. (2013). Reversible disruption of mSWI/SNF (BAF) complexes by the SS18-SSX oncogenic fusion in synovial sarcoma. Cell 153,71-85.
Kidder, B. L., and Zhao, K. (2014). Efficient library preparation for next-generation sequencing analysis of genome-wide epigenetic and transcriptional landscapes in embryonic stem cells. Methods Mol. Biol. 1150,3-20.
Langmead, B., and Salzberg, S. L. (2012). Fast gapped-read alignment with Bowtie 2. Nat. Methods 9,357-359.
Ma, P. C., Rould, M. A., Weintraub, H., and Pabo, C. O. (1994). Crystal structure of MyoD bHLH domain-DNA complex: perspectives on DNA recognition and implications for transcriptional activation. Cell 77,451-459.
Marguet, E. Forturre, P, (1998). Protection of DNA by salts against thermodegradation at temperatures typical for hyperthermophiles. Extremophiles, 2: 115-122.
McLean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat. Biotechnol. 28, 495-501.
Mei, S., Qin, Q., Wu, Q., Sun, H., Zheng, R., Zang, C. Zhu, M., Wu, J., Shi, X., Taking, L., et al (2017). Cistrome Data Browser: a data portal for Chip-Seq and chromatin accessibility data in human and mouse. Nucleic Acids Res. 45, D658—D662.
Miller, S. J., Jessen, W. J., Mehta, T., Hardiman, A., Sites, E., Kaiser, S., Jegga, A. G., Li, H., Upadhyaya, M., Giovannini, M., et al. (2009). Integrative genomic analyses of neurofibromatosis tumours identify SOX9 as a biomarker and survival gene. EMBO Mol. Med. 1, 236-248.
Miron, E., Oldenkamp, R., Brown, J. M., Pinto, D. M. S., Xu, C. S., Faria, A. R., Shaban, H. A., Rhodes, J. D. P., Innocent, C., de Ornellas, S., et al. (2020). Chromatin arranges in chains of mesoscale domains with nanoscale functional topography independent of cohesin. Sci. Adv. 6, eaba8811.
Nacev, B. A., Jones, K. B., Intlekofer, A. M., Yu, J. S. E., Allis, C. D., Tap, W. D., Ladanyi, M., and Nielsen, T. O. (2020). The epigenomics of sarcoma. Nat. Rev. Cancer 20, 608-623.
Pandey, P. R., Chatterjee, B., Olanich, M. E., Khan, J., Miettinen, M. M., Hewitt, S. M., and Barr, F. G. (2017). PAX3-FOXO1 is essential for tumour initiation and maintenance but not recurrence in a human myoblast model of rhabdomyosarcoma. J. Pathol. 241, 626-637.
Ptashne, M., and Gann, A. (1997). Transcriptional activation by recruitment. Nature 386, 569-577.
Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic Acids Res. 44, W160- W165.
Rao, S. S. P., Huang, S. C., Glenn St Hilaire, B., Engreitz, J. M., Perez, E. M., Kieffer-Kwon, K. R., Sanborn, A. L., Johnstone, S. E., Bascom, G. D., Bochkov, I. D., et al. (2017). Cohesin loss eliminates all loop domains. Cell 171, 305-320.e24
Romero, P., Obradovic, Z., Li, X., Garner, E. C., Brown, C. J., and Dunker, A. K. (2001). Sequence complexity of disordered protein. Proteins 42, 38-48.
Shern, J. F., Chen, L., Chmielecki, J., Wei, J. S., Patidar, R., Rosenberg, M., Ambrogio, L., Auclair, D., Wang, J., Song, Y. K., et al. (2014). Comprehensive genomic analysis of rhabdomyosarcoma reveals a landscape of fusion-positive and fusion-negative tumors. Cancer Discov. 4, 216-231.
Teves, S. S., An, L., Bhargava-Shah, A., Xie, L., Darzacq, X., and Tjian, R. (2018). A stable mode of bookmarking by TBP recruits RNA polymerase II to mitotic chromosomes. Elife 7, e35621.
Teves, S. S., An, L., Hansen, A. S., Xie, L., Darzacq, X., and Tjian, R. (2016). A dynamic mode of mitotic bookmarking by transcription factors. Elife 5, e22280.
Wachtel, M., and Schafer, B., (2018). PAX3-FOXO1: Zooming in on an “undruggable” target. Semin Cancer Biol., 50:115-123.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Wu, T. F., Yao, Y. L., Lai, I. L., Lai, C. C., Lin, P. L., and Yang, W. M. (2015). Loading of PAX3 to mitotic chromosomes is mediated by arginine methylation and associated with Waardenburg syndrome. J. Biol. Chem. 290, 20556-20564.
Xu, H. E., Rould, M. A., Xu, W., Epstein, J. A., Maas, R. L., and Pabo, C. O. (1999). Crystal structure of the human Pax6 paired domain-DNA complex reveals specific roles for the linker region and carboxy-terminal subdomain in DNA binding. Genes Dev. 13, 1263-1275.
Yan, C., Chen, H., and Bai, L. (2018). Systematic study of nucleosome-displacing factors in budding yeast. Mol. Cell 71, 294-305.e4.
Yan, J., Xu, L., Crawford, G., Wang, Z., and Burgess, S. M. (2006). The forkhead transcription factor FoxI1 remains bound to condensed mitotic chromosomes and stably remodels chromatin structure. Mol. Cell Biol. 26, 155-168.
Yang, J., Horton, J. R., Li, J., Huang, Y., Zhang, X., Blumenthal, R. M., and Cheng, X. (2019). Structural basis for preferential binding of human TCF4 to DNA containing 5-carboxylcytosine. Nucleic Acids Res. 47, 8375-8387.
Yohe, M. E., Gryder, B. E., Shern, J. F., Song, Y. K., Chou, H. C., Sindiri, S., Mendoza, A., Patidar, R., Zhang, X., Guha, R., et al. (2018). MEK inhibition induces MYOG and remodels super-enhancers in RAS-driven rhabdomyosarcoma. Sci. Transl Med. 10, eaan4470.
Zaret, K. S., and Carroll, J. S. (2011). Pioneer transcription factors: establishing competence for gene expression. Genes Dev. 25, 2227-2241.
Zaret, K. S., Watts, J., Xu, J., Wandzioch, E., Smale, S. T., and Sekiya, T. (2008). Pioneer factors, genetic competence, and inductive signaling: programming liver and pancreas progenitors from the endoderm. Cold Spring Harb Symp. Quant Biol. 73, 119-126.
Zheng, R., Wan, C., Mei, S., Qin, Q., Wu, Q., Sun, H., Chen, C. H., Brown, M , Zhang, X., Meyer, C. A., et al. (2019). Cistrome data browser: expanded datasets and new tools for gene regulatory analysis. Nucleic Acids Res. 47, D729—D735.
Zhou, B. R., Feng, H., Kale, S., Fox, T., Khant, H., de Val, N., Ghirlando, R., Panchenko, A. R., and Bai, Y. (2020). Distinct structures and dynamics of chromatosomes with different human linker histone isoforms. Mol. Cell 81, 166-182.e6.
Zhu, F., Farnung, L., Kaasinen, E., Sahu, B., Yin, Y., Wei, B., Dodonova, S. O., Nitta, K. R., Morgunova, E., Taipale, M., et al. (2018). The interaction landscape between transcription factors and the nucleosome. Nature 562, 76-81.

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method of identifying a plurality of regions in a genome that bind to a FOXO1-DNA Binding Domain Fusion Protein, comprising the steps of:
a) obtaining chromatin from a cell;
b) sonicating the chromatin;
c) isolating the chromatin by immunoprecipitation using an antibody that binds to FOXO1,
d) purifying the DNA from the immunoprecipitated chromatin;
e) amplifying and sequencing the DNA; and
f) analyzing the sequenced DNA to identify the locations in the genome of the cell that bind to the FOXO1-DNA Binding Domain Fusion Protein.

2. The method of claim 1, wherein the chromatin is sonicated for less than 15 minutes.

3. The method of claim 1, wherein the chromatin is incubated after sonication in a buffer having a salt concentration of at least 150 mM

4. The method of claim 1, wherein the antibody is the Cell Signaling Catalog #2880 antibody.

5. The method of claim 1, wherein the DNA Binding Domain is PAX3.

6. The method of claim 1, wherein the cell is a human cell, and the DNA is analyzed by alignment with human genome UCSC hg38.

7. The method of claim 1, wherein the cell is a cancer cell.

8. The method of claim 7, wherein the cancer cell is a rhabdomyosarcoma cancer cell.

9. The method of claim 1, wherein the chromatin is obtained from at least 1,000 cells.

10. The method of claim 1, further comprising the step of cross-linking the chromatin before obtaining it from the cell.

11. The method of claim 1, wherein one or more of the genomic regions are a portion of a gene.

12. The method of claim 1, wherein the method identifies at least 1,000 locations in the genome.

13. The method of claim 1, wherein the cells include human cells and rodent cells, wherein the rodent cells express wild-type FOXO1.

14. A method of treating a subject having rhabdomyosarcoma, by modulating the expression of a genomic region of a cancer cell identified by the method of claim 1.

15. The method of claim 14, wherein the DNA Binding Domain of the FOXO1-DNA Binding Domain Fusion Protein is PAX3.

16. The method of claim 14, wherein the expression of the genomic region is decreased.

17. The method of claim 16, wherein the expression is decreased by administering an effective amount of a siRNA to the subject.

18. The method of claim 14, wherein the genomic region is at least a portion of a gene.

19. The method of claim 14, wherein the expression of the genomic region is increased.
